(19) United States
(12) Patent Application Publication
(10) Pub. No.: US 2022/0181020 A1
(43) Pub. Date: Jun. 9, 2022

(54) SYSTEM AND METHOD FOR REMOTE PATIENT MONITORING

(71) Applicant: University Health Network, Toronto (CA)

(72) Inventors: Shafique Keshavjee, Toronto (CA); Marijana Zubrinic, Etobicoke (CA); Lukasz Brzozowski, Mississauga (CA); Xun Lin, Toronto (CA); Zigang Jimmy Qiu, Toronto (CA); An Wang, Thornhill (CA)

(21) Appl. No.: 17/598,179

(22) PCT Filed: Mar. 27, 2020
(86) PCT No.: PCT/IB2020/052964

§ 371 Date: Sep. 24, 2021

Related U.S. Application Data
(60) Provisional application No. 62/826,468, filed on Mar. 29, 2019.

(51) Int. Cl. G16H 40/67; G16H 40/20; G16H 50/20; A61B 5/11; A61B 5/00; G06V 20/52; G06V 10/25; G06V 10/28; G06V 10/44; G06V 10/70; H04N 5/232; G06F 3/01; G06F 40/58

(52) U.S. Cl. CPC G16H 40/67; G16H 40/20; G16H 50/20; A61B 5/1117; A61B 5/002; G06V 20/52; G06F 40/58; G06V 10/28; G06V 10/449; G06V 10/70; H04N 5/23299; G06F 3/013; G06V 10/25

(57) ABSTRACT

A system and method for providing and managing a remote patient monitoring (RPM) system. The method is implemented by a central server, an RPM client, and a networked monitoring device. The RPM client is a software program that is executed by a computing device that is connected to the server via a network. The networked monitoring device is implemented as a locator or a smart mobile cart. More specifically, the RPM system can provide a tele-monitor with the ability to remotely monitor multiple patients, control remote cameras, and address abnormal patient situations. The RPM system can enhance tele-monitor effectiveness by detecting patient motion and tracking tele-monitor alertness.

### CROSS-REFERENCE

This application claims the benefit of U.S. Provisional Patent Application No. 62/826,468, filed Mar. 29, 2019, and the entire contents of U.S. Provisional Patent Application No. 62/826,468 is hereby incorporated by reference.

### FIELD

Various embodiments are described herein that generally relate to a system and method for remote patient monitoring.

### BACKGROUND

The sickest of patients within healthcare settings are cared for in the intensive care unit (ICU) with continuous monitoring and 1:1 nursing care. The 1:1 ratio allows the nurse and other healthcare providers to dedicate 100% of their attention to the individual, instantly attend to their needs when required, and prevent adverse events. Outside of the ICU setting, there are various patient populations that are medically stable, but still require some form of continuous monitoring. A growing proportion of this population includes elderly patients who present with unique care needs, placing additional strain on critical healthcare resources. In 2017, 16.9% of Canada's population was 65 years or older and it is estimated to rise to 23% by 2030 (1,2). The reported co-morbidities and risks associated with hospitalization and/or surgery are different today than in previous decades due to our aging population. There has been a rise in postoperative delirium and confusion, as well as an increase in fall rates. Advances in surgical technology have provided patients with dementia the opportunity to undergo surgery where previously this may have not been possible.

An aging patient population is not unique to Canada and the impact is expected to have a major global effect on economic, social, and healthcare systems over the next 25-30 years (1). To address new challenges faced with treating older patients, most Western societies have implemented the bedside constant observer or sitter role. Sitters are usually non-nursing staff, i.e. typically nursing students, personal support workers, or security personnel, that provide around-the-clock, direct, 1:1 bedside observation of patients that are confused, delirious, and at risk for falls or other adverse events, with the intention to intervene and prevent patients from injuring themselves. While results have been favorable from a patient safety perspective, concerns remain regarding: (a) long-term sitter fatigue; and (b) growing sitter associated costs, forcing many healthcare organizations to question the sustainability of bedside 1:1 patient observation programs.

Given the current national and international demographic trends, there is expected a continuous increase in associated costs under the current bedside sitter scenario, and a greater need for an effective alternative constant monitoring solution.

### SUMMARY OF VARIOUS EMBODIMENTS

Various embodiments of a system and method for remote patient monitoring are provided according to the teachings herein.

According to one aspect of the invention, there is disclosed a computer-implemented method of managing a remote patient monitoring (RPM) system, wherein the method is implemented by a central server, an RPM client, and a networked monitoring device, and comprises: initializing the RPM system for remote monitoring of at least one patient location using a network and a networked monitoring device; receiving video data and physiological data over the network for the at least one patient location from the networked monitoring device; transmitting 2-way audio data over the network for the at least one patient location; displaying at least one viewport at the RPM client, the at least one viewport showing the video data and the physiological data for the at least one patient location; automatically detecting a patient situation requiring attention in the at least one patient location and indicating the patient situation on the RPM client; and receiving input at the RPM client of a response to the patient situation.

In at least one embodiment, initializing the RPM system further comprises: receiving a request from the RPM client to set up a user interface and display the user interface; providing camera data to the RPM client; updating the available/monitoring camera list with available and monitoring cameras; receiving a request from the RPM client to set up a viewport to monitor a specific camera from the available/monitoring camera list; connecting the RPM client to the specific camera using real-time streaming protocol (RTSP); receiving video frames from the specific camera and showing the video frames in the viewport; performing an adjustment of the specific camera according to a request from the RPM client where the adjustment includes adjusting one of a pan, tilt, and/or zoom setting for the specific camera; and sending audio input to a patient speaker associated with the specific camera where the audio input is received at the RPM client.

In at least one embodiment, the detected patient situation is that a patient is engaging in self harm and the method further comprises: signaling to the RPM client to instruct a tele-monitor to attempt to redirect the patient verbally; determining whether the redirection was successful; and when the redirection is not successful: signaling to the RPM client to instruct the tele-monitor to contact an assigned nurse to attend to the patient; and when the assigned nurse does not receive the contact, signaling to the RPM client to instruct the tele-monitor to contact a nursing station to attend to the patient; and determining that the patient situation has been responded to by receiving an indication that the assigned nurse or the nursing station has attended to the patient.

In at least one embodiment, the detected patient situation is that an SpO₂ level for a patient has dropped below an SpO₂ threshold level and the method further comprises: signaling to the RPM client to instruct the tele-monitor to contact an assigned nurse to attend to the patient; when the assigned nurse does not receive the contact, signaling to the RPM client to instruct the tele-monitor to contact a nursing station to attend to the patient; and determining that the patient situation has been responded to by receiving an indication that the assigned nurse or the nursing station has attended to the patient.

In at least one embodiment, the method further comprises: receiving video frames from a given camera; defining a reference background image including the patient from the video frames and defining a current image with the patient, the reference background image comprising background image pixels and the current image comprising current image pixels; creating a background model using the background image pixels using a mixture of Gaussian distributions, the background model having a background model distribution; classifying the current image pixels in the current image as background pixels or foreground pixels by calculating how close the current image pixels are from the background model distribution via Mahalanobis distance; collecting the current image pixels classified as foreground to generate a foreground image; applying a median blur filter to the foreground image to obtain a first filtered foreground image; applying a threshold filter to the first filtered foreground image to obtain a second filtered foreground binary image; applying an erosion filter to the second filtered foreground binary image to obtain a third filtered foreground binary image; applying a dilation filter to the third filtered foreground binary image to obtain a fourth filtered foreground binary image, the fourth filtered foreground binary image comprising 0-regions and 1-regions; finding borders of the 1-regions in the fourth filtered foreground binary image to generate contours; finding the contours that have areas larger than a predefined sensitivity value thereby defining found contours; overlaying the found contours onto the current image to obtain an overlaid current image; and displaying the overlaid current image at the RPM client.

In at least one embodiment, the method further comprises: receiving a video frame from a given camera; selecting a trained machine learning model for determining probabilities for pixels being associated with different classes in the video frame; calculating pixel class probabilities from the video frame using the trained machine learning model; assigning a pixel class label to each pixel using a highest class probability determined for each pixel; extracting class regions based on connected pixels that have the same pixel class label; calculating a bounding box around the connected regions; finding motion contours that have areas larger than a predefined sensitivity value thereby defining found motion contours; masking the found motion contours for bounding boxes for bed and person classes thereby defining a masked motion contour; overlaying the masked motion contour for a person on the video frame to obtain an overlay image; and displaying the overlay image at the RPM client.

In at least one embodiment, the trained machine learning model is an artificial neural network that is trained by supervised learning over datasets obtained from video data stored at the RPM system.

In at least one embodiment, the incident comprises a patient falling out of bed and the method comprises use machine learning methods to predict when the incident will take place based on the video data received at the RPM client.

In at least one embodiment, the incident comprises a low patient SpO₂ level below an SpO₂ threshold, and the method comprises use machine learning methods to predict when the incident will take place based on the physiological data received at the RPM client.

In at least one embodiment, the method further comprises: receiving gaze data from the RPM client on a gaze direction of the tele-monitor determined using an eye tracker, the gaze data including gaze direction vectors; performing screen calibration of a screen of the RPM client; calculating a screen pixel location from the gaze direction vectors; identifying when the gaze direction is outside of the viewport based on the screen pixel location; and when the gaze direction is outside of the viewport longer than a gaze alert timer threshold, providing an audio and or video alert to the tele-monitor to prompt the tele-monitor to view the viewport.

In at least one embodiment, the method further comprises: translating between first speech input received by the RPM client and second speech input received from the at least one patient location using natural language processing, speech recognition, and speech synthesis so that communication at the RPM client and the least one patient location is in different languages spoken by individuals at both the RPM client and the least one patient location.

In at least one embodiment, the at least one networked monitoring device comprises at least one of a locator that is used to configure a subnet for the patient locations at one physical location and a mobile patient monitoring cart that is used to create its own subnet to connect to the network.

In at least one embodiment, the mobile patient monitoring cart comprises a camera, a speaker, and at least one physiological measuring device incorporated into one mobile unit and the method further comprises deploying the mobile patient monitoring cart to a different patient location.

In at least one embodiment, the method further comprises: employing multiple networked monitoring devices and multiple RPM clients to scale the remote monitoring to cover patient locations in different locations within one building or in different locations in different buildings including a patient home.

In at least one embodiment, the network comprises at least one of a wired subnet and a wireless subnet that uses at least one of dynamic IP and static IP.

In another aspect, there is disclosed a system for remote patient monitoring (RPM), the system comprising: a server comprising a data store and at least one processor coupled to the data store; an RPM client that is a software program that is executed by a computing device that is connected to the server via a network; and a networked monitoring device that is connected to the server and the computing device having the RPM client via the network; wherein the server is configured to initialize the RPM system for remote monitoring of at least one patient location using the network, and wherein the RPM client is configured to receive video data and physiological data over the network for the at least one patient location via the networked monitoring device; transmit 2-way audio data over the network for the at least one patient location; display at least one viewport at the RPM client, the at least one viewport showing the video data and the physiological data for the at least one patient location; automatically detect a patient situation requiring attention in the at least one patient location and indicate the patient situation on the RPM client; and receive input from the RPM client of a response to the patient situation.

In at least one embodiment, the server is configured to initialize the RPM system by: receiving a request from the RPM client to set up a user interface and display the user interface; providing camera data to the RPM client; updating the available/monitoring camera list with available and monitoring cameras; receiving a request from the RPM client to set up a viewport to monitor a specific camera from the available/monitoring camera list; connecting the RPM client to the specific camera using real-time streaming protocol (RTSP); receiving video frames from the specific camera and showing the video frames in the viewport; performing an adjustment of the specific camera according to a request from the RPM client where the adjustment includes adjusting one of a pan, tilt, and/or zoom setting for the specific camera; and sending audio input to a patient speaker associated with the specific camera where the audio input is received at the RPM client.

In at least one embodiment, the detected patient situation is that a patient is engaging in self harm and the computing device is configured to execute instructions to: provide a signal at the RPM client to instruct a tele-monitor to attempt to redirect the patient verbally; determine whether the redirection was successful; and when the redirection is not successful: signal to the RPM client to instruct the tele-monitor to contact an assigned nurse to attend to the patient; and when the assigned nurse does not receive the contact, signal to the RPM client to instruct the tele-monitor to contact a nursing station to attend to the patient; and determine that the patient situation has been responded to by receiving an indication that the assigned nurse or the nursing station has attended to the patient.

In at least one embodiment, the detected patient situation is that an SpO₂ level for a patient has dropped below an SpO₂ threshold level and the computing device is configured to execute instructions to: signal to the RPM client to instruct the tele-monitor to contact an assigned nurse to attend to the patient; when the assigned nurse does not receive the contact, signal to the RPM client to instruct the tele-monitor to contact a nursing station to attend to the patient; and determine that the patient situation has been responded to by receiving an indication that the assigned nurse or the nursing station has attended to the patient.

In at least one embodiment, the computing device is configured to execute instructions to: receive video frames from a given camera; define a reference background image including the patient from the video frames and defining a current image with the patient, the reference background image comprising background image pixels and the current image comprising current image pixels; create a background model using the background image pixels using a mixture of Gaussian distributions, the background model having a background model distribution; classify the current image pixels in the current image as background pixels or foreground pixels by calculating how close the current image pixels are from the background model distribution via Mahalanobis distance; collect the current image pixels classified as foreground to generate a foreground image; apply a median blur filter to the foreground image to obtain a first filtered foreground image; apply a threshold filter to the first filtered foreground image to obtain a second filtered foreground binary image; apply an erosion filter to the second filtered foreground binary image to obtain a third filtered foreground binary image; apply a dilation filter to the third filtered foreground binary image to obtain a fourth filtered foreground binary image, the fourth filtered foreground binary image comprising 0-regions and 1-regions; find borders of the 1-regions in the fourth filtered foreground binary image to generate contours; find the contours that have areas larger than a predefined sensitivity value thereby defining found contours; overlay the found contours onto the current image to obtain an overlaid current image; and display the overlaid current image at the RPM client.

In at least one embodiment, the computing device is configured to execute instructions to: receive a video frame from a given camera; select a trained machine learning model for determining probabilities for pixels being associated with different classes in the video frame; calculate pixel class probabilities from the video frame using the trained machine learning model; assign a pixel class label to each pixel using a highest class probability determined for each pixel; extract class regions based on connected pixels that have the same pixel class label; calculate a bounding box around the connected regions; find motion contours that have areas larger than a predefined sensitivity value thereby defining found motion contours; mask the found motion contours for bounding boxes for bed and person classes thereby defining a masked motion contour; overlay the masked motion contour for a person on the video frame to obtain an overlay image; and display the overlay image at the RPM client.

In at least one embodiment, the trained machine learning model is an artificial neural network that is trained by supervised learning over datasets obtained from video data stored at the RPM system.

In at least one embodiment, the incident comprises a patient falling out of bed and the computing device is configured to execute machine learning methods to predict when the incident will take place based on the video data received at the RPM client.

In at least one embodiment, the incident comprises a low patient SpO₂ level below an SpO₂ threshold, and the computing device is configured to execute machine learning methods to predict when the incident will take place based on the physiological data received at the RPM client.

In at least one embodiment, the computing device is configured to execute instructions to: receive gaze data from the RPM client on a gaze direction of the tele-monitor determined using an eye tracker, the gaze data including gaze direction vectors; perform screen calibration of a screen of the RPM client; calculate a screen pixel location from the gaze direction vectors; identify when the gaze direction is outside of the viewport based on the screen pixel location; and when the gaze direction is outside of the viewport longer than a gaze alert timer threshold, provide an audio and or video alert to the tele-monitor to prompt the tele-monitor to view the viewport.

In at least one embodiment, the computing device is configured to execute instructions to: translate between first speech input received by the RPM client and second speech input received from the at least one patient location using natural language processing, speech recognition, and speech synthesis so that communication at the RPM client and the least one patient location is in different languages spoken by individuals at both the RPM client and the least one patient location.

In at least one embodiment, the at least one networked monitoring device comprises at least one of a locator that is used to configure a subnet for the patient locations at one physical location and a mobile patient monitoring cart that is used to create its own subnet to connect to the network.

In at least one embodiment, the mobile patient monitoring cart comprises a camera, a speaker, and at least one physiological measuring device incorporated into one mobile unit and the mobile patient monitoring cart is deployed to a different patient location.

In at least one embodiment, the system further comprises multiple networked monitoring devices and multiple RPM clients to scale the remote monitoring to cover patient locations in different locations within one building or in different locations in different buildings including a patient home.

In at least one embodiment, the network comprises at least one of a wired subnet and a wireless subnet that uses at least one of dynamic IP and static IP.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A illustrates an example embodiment of a remote patient monitoring system in accordance with the teachings herein showing camera discovery and server end points.

FIG. 1B shows an example embodiment of a server that can be used with the remote monitoring system of FIG. 1A.

FIG. 1C shows a flow chart of an example embodiment of a method of managing the remote monitoring system of FIG. 1A.

FIG. 2 shows an example of a 2×2 layout of a client application in which a user can select single view, 1×2, 2×2, and 2×3 views in the top bar, where patient faces were covered by rectangles for their privacy.

FIG. 3 shows an example of a 2×3 layout of the client application, where patient faces were covered by rectangles for their privacy.

FIG. 4 shows an example of a dialog window for linking a camera to a viewport in the layout and in which a user can specify the associated camera, risk factors, and patient information.

FIG. 5 shows an example of a camera settings window specifying encoding type, resolution, and credentials.

FIG. 6 shows an example of a logging window for users to log problems associated with camera and site.

FIG. 7 shows an example of motion highlight overlay on video in which detected motion is highlighted with a red outline, where the patient's face was whited out for their privacy.

FIG. 8 is a chart of the pre-lung transplant (wait list) mortality rate for 2004 to 2019.

FIG. 9 shows a flow chart of an example embodiment of a method of controlling a camera in the remote monitoring system of FIG. 1A.

FIG. 10 shows a flow chart of an example embodiment of a method of managing cameras in the remote monitoring system of FIG. 1A.

FIG. 11 shows a flow chart of an example embodiment of a method of automating a self-harm response in the remote monitoring system of FIG. 1A.

FIG. 12 shows a flow chart of an example embodiment of a method of automating an oxygen saturation abnormality response in the remote monitoring system of FIG. 1A.

FIG. 13 shows a flow chart of an example embodiment of a method of detecting and displaying patient motion in the remote monitoring system of FIG. 1A.

FIG. 14 shows a flow chart of an example embodiment in which machine learning is applied in a method of detecting and displaying patient motion in the remote monitoring system of FIG. 1A.

FIG. 15 shows a flow chart of an example embodiment of a method of gaze alertness tracking in the remote monitoring system of FIG. 1A.

FIG. 16 shows a flow chart of an example embodiment of a method of initializing and communicating with a mobile cart configuration in the remote monitoring system of FIG. 1A.

FIG. 17 shows an example screen view on an RPM client that demonstrates motion detection of an observer raising a hand.

FIG. 18 shows an example screen view on an RPM client that demonstrates motion detection of an observer getting up.

FIG. 19 shows an example screen view on an RPM client that demonstrates eye tracking.

FIG. 20 shows a chart of bedside constant observer hours for April 2018 to March 2019 and the three-year period from April 2014 to March 2017.

FIG. 21 shows an example decision support tool for initiation, continuation, and discontinuation of remote monitoring.

FIG. 22 shows an example list of consideration factors for appropriateness of remote monitoring.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems, or methods having all of the features of any one of the devices, systems, or methods described below or to features common to multiple or all of the devices, systems, or methods described herein. It is possible that there may be a device, system, or method described herein that is not an embodiment of any claimed subject matter. Any subject matter that is described herein that is not claimed in this document may be the subject matter of another protective instrument, for example, a continuing patent application, and the applicants, inventors, or owners do not intend to abandon, disclaim, or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical signal, electrical connection, wireless connection, or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5%, or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

It should also be noted that the use of the term “window” in conjunction with describing the operation of any system or method described herein is meant to be understood as describing a user interface for performing initialization, configuration, or other user operations.

The example embodiments of the devices, systems, or methods described in accordance with the teachings herein may be implemented as a combination of hardware and software. For example, the embodiments described herein may be implemented, at least in part, by using one or more computer programs, executing on one or more programmable devices comprising at least one processing element and at least one storage element (i.e., at least one volatile memory element and at least one non-volatile memory element). The hardware may comprise input devices including at least one of a touch screen, a keyboard, a mouse, buttons, keys, sliders, and the like, as well as one or more of a display, a printer, and the like depending on the implementation of the hardware.

It should also be noted that there may be some elements that are used to implement at least part of the embodiments described herein that may be implemented via software that is written in a high-level procedural language such as object oriented programming. The program code may be written in MATLAB, C, C#, C⁺⁺, Java, JavaScript, or any other suitable programming language and may comprise modules or classes, as is known to those skilled in object oriented programming. Alternatively, or in addition thereto, some of these elements implemented via software may be written in assembly language, machine language or firmware as needed. In either case, the language may be a compiled or interpreted language.

At least some of these software programs may be stored on a computer readable medium such as, but not limited to, a ROM, a magnetic disk, an optical disc, a USB key, and the like that is readable by a device having a processor, an operating system, and the associated hardware and software that is necessary to implement the functionality of at least one of the embodiments described herein. The software program code, when read by the device, configures the device to operate in a new, specific, and predefined manner (e.g., as a specific purpose computer) in order to perform at least one of the methods described herein.

At least some of the programs associated with the devices, systems, and methods of the embodiments described herein may be capable of being distributed in a computer program product comprising a computer readable medium that bears computer usable instructions, such as program code, for one or more processing units. The medium may be provided in various forms, including non-transitory forms such as, but not limited to, one or more diskettes, compact disks, tapes, chips, cloud storage, and magnetic and electronic storage. In alternative embodiments, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

In accordance with the teachings herein, there are provided various embodiments for systems and methods for implementing improved Remote Patient Monitoring (RPM). A remote patient monitoring system addresses the need for an effective alternative constant monitoring solution. For example, an RPM system may be used to monitor high-risk patients 24/7 while reducing direct observation costs and reducing patient mortality/morbidity. Accordingly, an RPM system presents an attractive alternative for healthcare organizations to ensure high-risk patients, such as the elderly population, remain safe. This RPM technology includes a small wireless or wired camera with speakers and a microphone mounted on wheels that can be transported to the bedside. The patient is continuously monitored through a video monitoring system and verbally redirected through the microphone and speakers by a patient observation technician from a remote location in the hospital or other medical or care facility. If the technician cannot verbally redirect the patient or the patient is demonstrating unsafe behaviors, the technician may immediately alert the nurse on a dedicated phone to attend to the patient. A specially trained technician can watch multiple patients simultaneously, with one technician monitoring anywhere from 4-18 patients at one time as reported in pilot studies in the United States (4, 5, 6).

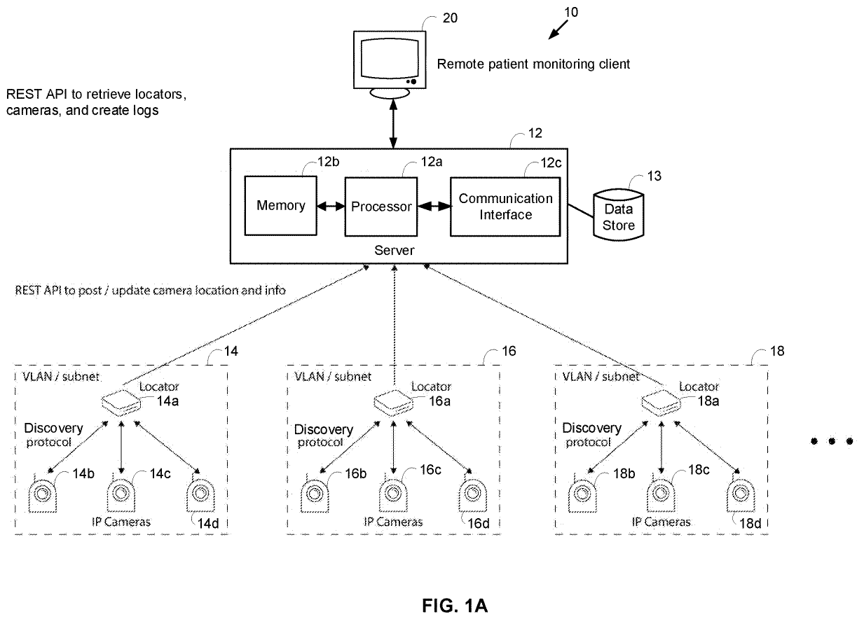

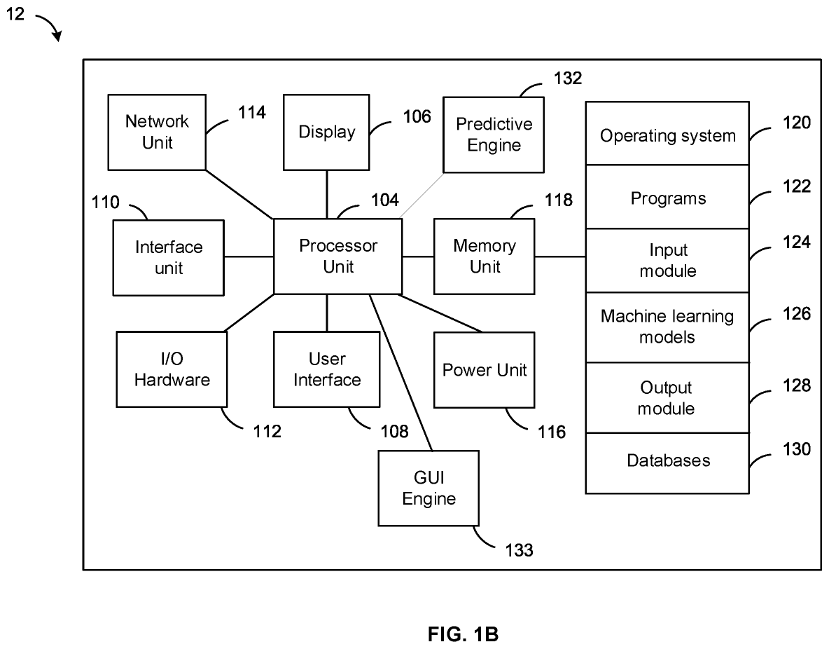

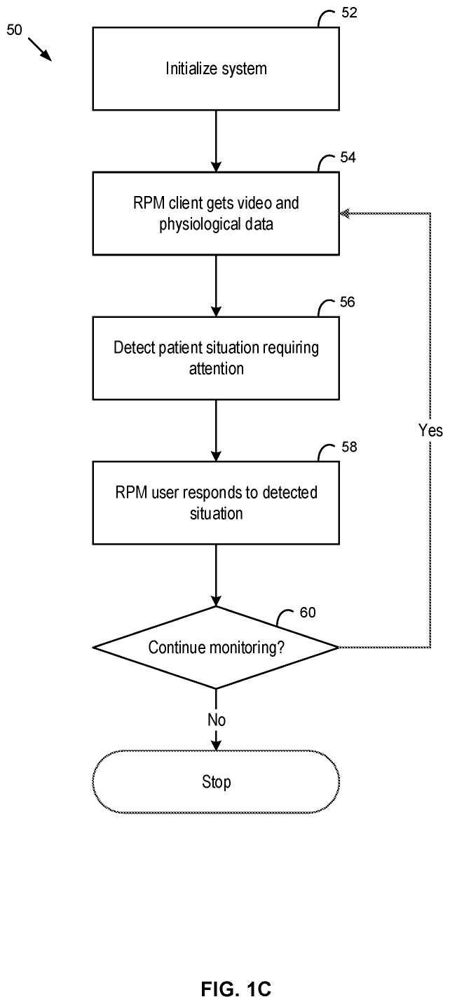

Reference is first made to FIG. 1A, showing an example embodiment of a remote patient monitoring system 10 showing camera discovery and server end points.

The system 10 includes a server 12 for controlling the operation of the system 10. The server 12 has a processor 12a, a memory 12b, and a communication interface 12c. The server 12 is coupled with a data store 13, which can store data generated and/or received by the server 12. The data store 13 may also store one or more databases with various hardware and/or patient specific information. The server 12 may generate data relating to site, floor, camera, and IP info. The server 12 communicates with a remote patient monitoring (RPM) client 20, which is a client application that is used by a technician (e.g., user, observer, tele-monitor, or operator) for remote observation and communication from a remote station to one of the patient rooms that has a server end point that are organized as subnets. For example, the server 12 communicates with subnets 14, 16, and 18. While three subnets 14, 16, and 18 are shown in FIG. 1A, this is for illustration purposes only as there may be more or fewer subnets as needed or desired. For the sake of readability, reference will be made to subnet 14 only, although such reference should be interpreted as meaning any one or more subnets 14, 16 and 18. The RPM client 20 may be operated on a desktop computer or another suitable computing device such as a laptop, a tablet or a smart phone.

The server 12 is implemented as a server for camera discovery across different subnets 14, 16 and 18 on a network. The subnet 14 may be implemented as Virtual Local Area Networks (VLANs). The subnet 14 comprises a networked monitoring device 14a (shown in FIG. 1A as “Locator 14a”) and cameras 14b, 14c, and 14d. Three cameras 14b, 14c, and 14d are shown in FIG. 1A for illustration purposes only; there may be more or fewer cameras as needed or desired. For the sake of readability, reference will be made to camera 14b only, although such reference should be interpreted as meaning any one or more cameras. The subnet 14 communicates with the server 12 through the networked monitoring device 14a.

The network may be a simple network (e.g., a single network with one type of IP), may be a complex network (e.g., both wired/wireless networks, different subnets, multiple VLANs, multiple sites, different visibility, dynamic IP, and static IP), or in between. For example, the network may be all wired, all wireless, or both wired and wireless. The network may include wired subnets 14, wireless subnets 14, or both wired and wireless subnets 14. Similarly, each subnet 14 may be all wired, all wireless, or both wired and wireless. Also, for example, a portion or all of the network may use dynamic IP, static IP, or both. Similarly, each subnet 14 may use dynamic IP, static IP, or both.

In at least one embodiment, the server 12 automates texting and calling (e.g., to a nursing station) via a service that is accessible by or pushed to the RPM client 20. Alternatively, or in addition, this automation can be integrated into the RPM client 20. This automation can advantageously render communication faster than an observer having to manually call via a hospital phone.

The networked monitoring device 14a may be implemented as a locator, or as a patient monitoring device, such as a mobile remote patient monitoring cart (also referred to herein as a “mobile cart”), or both.

In at least one embodiment, the networked monitoring device 14a acts as a locator. As a locator, the networked monitoring device 14a is an embedded device or computer that sends a User Datagram Protocol (UDP) broadcast across the same subnet 14. A camera 14b on the subnet 14 responds with its name, IP, port, MAC address, and video encoding format. The networked monitoring device 14a communicates with the camera 14b (e.g., an IP camera) through the UDP broadcast and updates the list of discovered cameras 14b to the server 12 at a periodic interval. The camera 14b may be a mobile camera unit having a pan-tilt-zoom (PTZ) camera with Ethernet/WiFi connectivity, two-way audio, a built-in microphone, and stereo speakers. The camera 14b may be, for example, a webcam, a built-in webcam, or an integrated camera.

In at least one embodiment, the networked monitoring device 14a acts as a mobile cart. As a mobile cart, the networked monitoring device 14a creates its own subnet 14 and acts as a gateway for network traffic between the mobile cart (which may include a camera 14b and other peripheral devices) and the server 12. The networked monitoring device 14a may be discoverable by the server 12 and networked together to enable RPM for a given site. The networked monitoring device 14a may have an embedded computing device (e.g., mini-computer, wireless router, network bridge) on the mobile cart which allows it to be discoverable to the system 10 outside of its subnet 14. The networked monitoring device 14a may also enable other measuring devices to be connected to the mobile cart to send patient physiological data to the server 12.

In at least one embodiment, the networked monitoring device 14a uses a one-to-one configuration with a camera 14b and at least one physiological measurement device. Accordingly, the networked monitoring device 14a may act as a locator that is paired with a single camera 14b and deployed on a mobile cart. The networked monitoring device 14a enables pass-through of network traffic to the camera 14b and broadcasting of its network information. Advantageously, the one-to-one configuration is more agnostic to constraints of the network infrastructure. For example, a single physical site or patient floor may be partitioned into multiple subnets due to incremental changes over the years or legacy integration reasons. A nursing station area may only be part of one subnet where some patient rooms may belong to others. In this topology, a single locator with discovery protocol via UDP broadcast would not be able to identify cameras in all patient rooms. The one-to-one configuration, in contrast, advantageously enables a single networked monitoring device 14a acting as a mobile cart to send its own network information to the server 12. The RPM client 20 can then connect to the cameras 14b the same way as the networked monitoring device 14a would pass-through network traffic to and from the cameras 14b. Advantageously, the one-to-one configuration can ensure that the RPM client 20 is able to connect to the camera 14b regardless of what the network infrastructure may be.

In at least one embodiment, the networked monitoring device 14a uses a one-to-many configuration. Accordingly, the networked monitoring device 14a may act as a single locator that is used to discover many cameras 14b and associated physiological measurement devices on the same subnet 14 via UDP broadcast. This advantageously can provide better resource management at, for example, smaller clinics or enterprises where sites are partitioned by subnets that mirrors physical partitioning.

In at least one embodiment, the system 10 may also include cloud voice calling capabilities (e.g., for nurses when handling escalations). In such cases, the communication interface 12c is connected to a cloud system via the Internet (both not shown).

In at least one embodiment, the RPM client 20 may also include gaze and head tracking hardware and/or software to determine observer (e.g., tele-monitor) attention.

In at least one embodiment, the networked monitoring device 14a is a small embedded Linux system deployed across hospitals according to subnet 14 division. The networked monitoring device 14a runs the camera discovery protocol periodically (e.g., every 30 seconds) by broadcasting a request message across the subnet 14. The cameras 14b listen for the request message and reply with an acknowledgement message that includes the camera name, the camera IP address, port, device MAC address, and the video encoding format that is used by the camera. The networked monitoring device 14a then sends the list of discovered cameras to the server 12, which aggregates and groups cameras 14b by the subnets 14 (or networked monitoring devices 14a). These lists are displayed and contextualized to the RPM client 20 according to buildings, floors, and patient units. Alternatively, an Ethernet bridge or camera with scripting capabilities can broadcast its IP and meta info (e.g., name, video encoding) to the server 12 once connected to the network.

In at least one embodiment, the server 12 provides a representational state transfer (REST) application program interface (API) with endpoints for networked monitoring devices 14a to create/update discovery information and a client application on the RPM client 20 to get camera lists. The server 12 also enables continuous real-time communication to the client application via WebSockets to push message notifications pending on status such as dispatch calls to nursing when handling observed incidents that require escalation. Nurses may receive these messages on a cell phone or a wireless hospital phone with a hospital extension number. The REST

API endpoint can also interface with cloud voice call services for automating dispatch calls and responses. The REST API endpoints include requests for at least one of locators, logs, cameras, SpO₂ (peripheral oxygen saturation) devices, voice call notification, as well as login and authentication.

In at least one embodiment, the locator endpoints allow updates and retrieval of camera lists based on discovery across subnets. The locator endpoints include one or more of the following software modules:
GET locator modules which retrieve a complete list of locators from a database (with the option to retrieve only active locators);
POST locator modules which create a new locator associated with a location, including name, building, floor, IP address, and current list of cameras;
GET locator modules specifying site, floor, unit which retrieve a specific locator according to a queried site; and
PUT locator modules specifying site, floor, unit which update a specific locator according to a queried site.

In at least one embodiment, the log endpoints allow error reporting and retrieval by floor, room, and camera. The log endpoints include one or more software modules including:
GET log modules which retrieve a complete list of logs from a database as well as filtering by query parameters; and
POST log modules which create a new log associated with camera, floor, room, description of problem, and time of incident.

In at least one embodiment, the camera endpoints include one or more software modules including:
GET camera modules which retrieve a complete list of cameras in a database as well as according to query by name;
POST camera modules which create a new camera, including name, resolution, vendor, model, note, and associated oximeter;
PUT camera modules which update a camera according to name; and
DELETE camera modules which delete a camera from a database according to name.

In at least one embodiment, the SpO₂ endpoints include one or more software modules including:
GET SpO₂ modules which retrieve a list of oximeters from a database as well as according to name;
POST SpO₂ modules which create a new oximeter device, including name and IP address; and
PUT SpO₂ modules which update an existing oximeter, queried by name.

In at least one embodiment, the touch-to-call notification endpoints include one or more software modules including:
GET notification modules which parse request parameters (name, digit, from, to, message) and encode it as a message to send to a desktop client via a WebSocket.

In at least one embodiment, the authentication endpoints include one or more software modules including:
POST register user modules which register user using supplied username and password;
POST login user modules which authenticate user based on credentials; and
POST login locator modules which authenticate locator based on credentials.

Referring now to FIG. 1B, shown therein is a block diagram of an example embodiment of the server 12. The system server 12 may run on a single computer, including a processor unit 104, a display 106, a user interface 108, an interface unit 110, input/output (I/O) hardware 112, a network unit 114, a power unit 116, and a memory unit (also referred to as “data store”) 118. In other embodiments, the server 12 may have more or less components but generally function in a similar manner. For example, the server 12 may be implemented using more than one computing device.

The processor unit 104 may include a standard processor, such as the Intel Xeon processor, for example. Alternatively, there may be a plurality of processors that are used by the processor unit 104 and these processors may function in parallel and perform certain functions. The display 106 may be, but not limited to, a computer monitor or an LCD display such as that for a tablet device. The user interface 108 may be an Application Programming Interface (API) or a web-based application that is accessible via the network unit 114. The network unit 114 may be a standard network adapter such as an Ethernet or 802.11x adapter.

The processor unit 104 may execute a predictive engine 132 that functions to provide predictions by using machine learning models 126 stored in the memory unit 118. The predictive engine 132 may build a predictive algorithm through machine learning. The training data may include, for example, recorded video and audio data, as well as physiological data including at least SpO₂ data and/or motion data. The predictive algorithm uses these data to predict whether a patient can be expected to behave erratically or whether a clinical event may occur. For example, the predictive engine 132 then executes the predictive algorithm when monitoring patients.

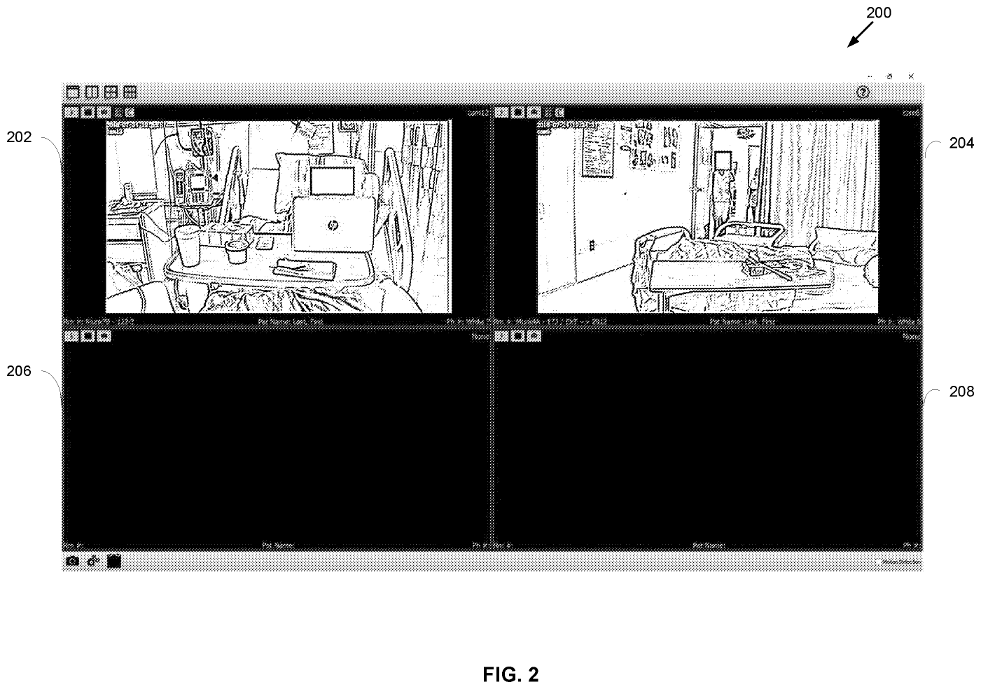

The processor unit 104 can also execute a graphical user interface (GUI) engine 133 that is used to generate various GUIs, some examples of which are shown (e.g., windows and dialog boxes shown in FIGS. 2 to 7) and described herein. The GUI engine 133 provides data according to a certain layout for each user interface and also receives data input or control inputs from a user. The GUI then uses the inputs from the user to change the data that is shown on the current user interface, or changes the operation of the system 10 which may include showing a different user interface.

The memory unit 118 may store the program instructions for an operating system 120, program code 122 for other applications, an input module 124, a plurality of machine learning models 126, output module 128, and databases 130. The machine learning models 126 may include, but are not limited to, image recognition and categorization algorithms based on deep learning models and other approaches.

In at least one embodiment, the machine learning models 126 include a combination of convolutional and recurrent neural networks. Convolutional neural networks (CNNs) are designed to recognize images, patterns. CNNs perform convolution operations, which, for example, can be used to classify regions of an image, and see the edges of an object recognized in the image regions. Recurrent neural networks (RNNs) can be used to recognize sequences, such as text, speech, and temporal evolution, and therefore RNNs can be applied to a sequence of data to predict what will occur next. Accordingly, a CNN may be used to read what is happening on a given image at a given time (e.g., the edge of the bed has been crossed by a person), while an RNN can be used to provide a warning message such as “based on what has been learned from other images, it is predicted that the patient may be moving towards the edge of the bed” or “based on what has been learned from physiological data, the vitals suggest that a clinical event may be coming soon”.

The programs 122 comprise program code that, when executed, configures the processor unit 104 to operate in a particular manner to implement various functions and tools for the system 10.

Referring now to FIG. 1C, shown therein is a flow chart of an example embodiment of a method 50 of managing the RPM system of FIG. 1A.

At act 52, the server 12 initializes the system 10. The RPM client 20 may be initialized at this time too. Alternatively, or in addition thereto, the RPM client 20 may be on and waiting to receive data from the server 12.

At act 54, the RPM client 20 obtains video data (and possibly audio data) and physiological data directly from the cameras 14b and physiological sensors or physiological monitoring devices. Alternatively, or in addition, the RPM client 20 may obtain the physiological data from a gateway connected to other physiological measuring devices or through a hospital HL7 server. The video is generated by the cameras 14b. The physiological data include information on a patient's vitals such as, but not limited to, heart rate, blood pressure, SpO₂, and temperature, for example. The physiological data may be obtained by using various measuring devices, instruments, sensors, monitors, or meters.

At act 56, the RPM client 20 detects a patient situation requiring attention based at least in part on the video data received from the cameras 14b. Alternatively, or in addition thereto, the detection of the patient situation requiring attention may be based on the physiological data. The patient situations requiring attention include, but are not limited to: the patient engaging in self-harm; the patient's SpO₂ level dropping below a threshold (e.g., a preset threshold, or a threshold set by the RPM client 20, or determined by machine learning); and one of sudden movement, excessive movement, or no movement for a certain duration of time by the patient.

At act 58, the RPM client 20 receives response data in response to the detected situation. The response data may include input by a user (e.g., a tele-monitor) of the RPM client 20 or data indicating that the user of the RPM client 20 has initiated a response to the detected situation. The user may respond to the situation, for example, by calling an assigned nurse by phone or sending an electronic message to the assigned nurse, and such a call or message may signal to the server 12 that a response has been initiated. The call or text may be input at the RPM client 20 and sent to the server 12 as an electronic communication message.

At act 60, an operator of the RPM client 20 (or a clinical team or tele-monitor) determines whether to continue monitoring. The determination may be entered into the RPM client 20 and sent to the server 12 as control data. The determination may be based, for example, on: the user inputting to the RPM client 20 that the situation is resolved or not; the nurse signaling to the system 10 that the situation has been resolved or needs further attention; or the RPM client 20 receiving video data from the camera 14b or physiological data from one or more sensors that the situation has been resolved or not. If the user determines to continue monitoring, the method 50 returns to act 54. If the user determines not to continue monitoring, the method 50 ends.

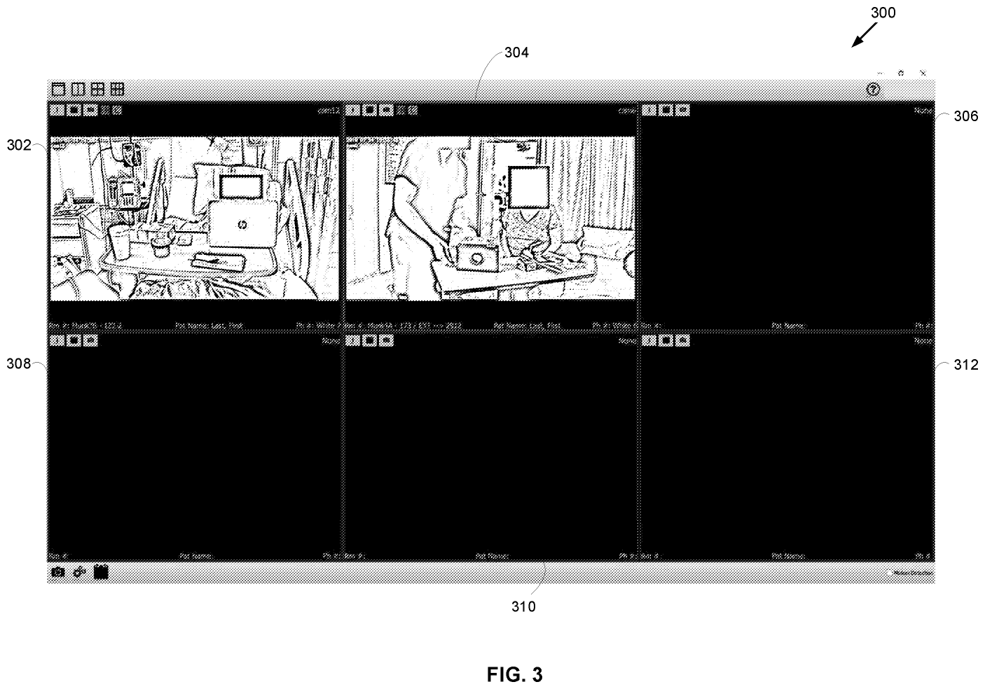

Referring now to FIGS. 2 and 3, show therein are examples of different layouts of a client application in which a user can select a single view, 1×2, 2×2, and 2×3 views in the top bar. Other types of views may also be used in other embodiments. The client application is provided as part of the RPM client 20.

FIG. 2 shows an example of a 2×2 layout 200 of the client application. In the 2×2 layout 200, a first viewport 202 of a room shown by a camera 14b is seen in one window pane. A second viewport 204 of a room shown by another camera 14c is seen in a second window pane. The other window panes 206 and 208 are black because no other camera feeds are sent to the client application.

FIG. 3 shows an example of a 2×3 layout 300 of the client application. in the 2×3 layout 300, a first viewport 302 of a room shown by a camera 14b is seen in one window pane. A second viewport 304 of a room shown by another camera 14c is seen in a second window pane. The other window panes 306, 308, 310, and 312 are black because no other camera feeds are sent to the client application.

In at least one embodiment, the client application runs on the RPM client 20 and enables a remote observer (e.g., a user, also known as a tele-monitor, observer, or operator) to connect to cameras 14b in patient rooms and stream audio/video from the camera 14b and microphone to receive video data and audio data from the patient room. The observer can choose from different layouts to observe multiple patients simultaneously. Overlays of at least one of patient name, site, floor, room, and dispatch call information may be overlaid over the displayed video.

The client application connects to the server 12 via REST API endpoints to retrieve a list of cameras 14b with identifying data, technical data, and location data. The technical data may include, for example, a name, an IP address, a port, a MAC address, a video encoding format, and video resolution. The port may be an Ethernet port, or a number associated with communicating with the IP; for example, many HTTP servers serve web pages on port 80. The location data may include, for example, a site, floor, or unit. The observer may associate a camera 14b, the patient being observed, and the nurse along with their dispatch phone information for handling escalations by entering data into certain fields in the client application, which is sent to the server 12. The association can be edited and updated based on a change of at least one of patient and/or nurse. Some or all of the data described above may be sorted for future auditing.

The observer may interact with the camera 14b through a main user interface and viewports of the client application. The observer can view, enter, and update data such as, but not limited to, at least one of:
Camera data: one or more of vendor, model, resolution, and notes;
Camera reports: error logging in the context of one or more of a camera, site, floor, and unit;
Camera settings: for associating a camera with one or more of a site, floor, unit, patient, and dispatch nurse (stored locally at the server 12 for fast reboot purposes except for patient name which is stored in volatile memory);
Physiological data: patient's vitals such as, but not limited to, at least one of heart rate, blood pressure, SpO₂, and temperature, etc.;
Application settings: one or more of login credentials, server URL, network adapter selection, motion detection sensitivity, and default camera login; and
General reports: which is an application related error logging.

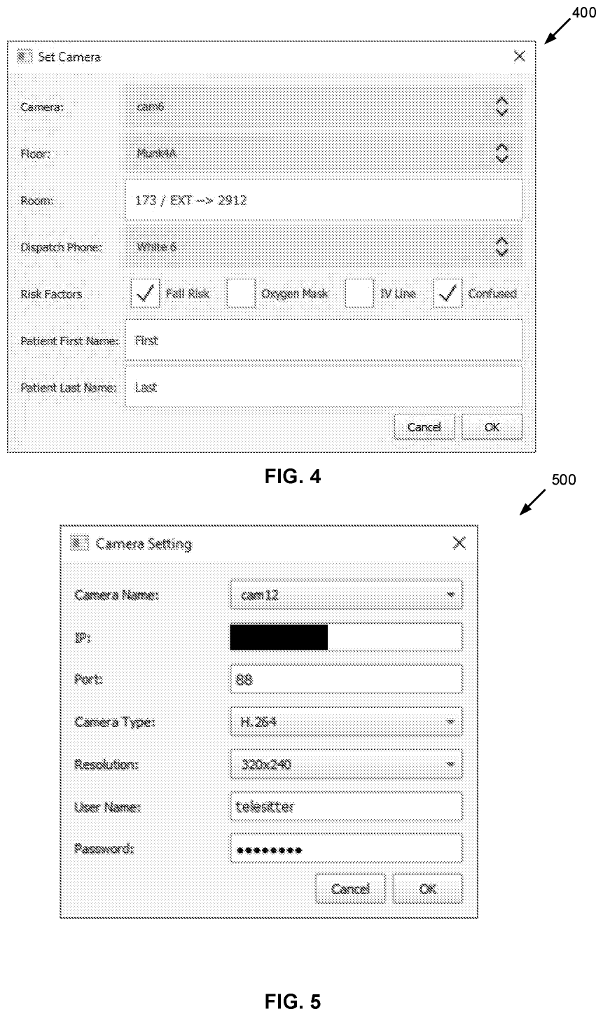

The layouts for the viewports include, for example, 1×1, 1×2, 2×2, and 2×3. Clicking on a viewport sets the active viewport and camera 14b. The border of the active viewport is highlighted to indicate that it has been selected to be active. The observer can then engage in camera controls and voice communication with the associated camera 14b. Appropriate icons that indicate specific actions (such as push-to-talk active and sound) may be overlaid on top of the video. The RPM client 20 may also include settings and dialog windows as shown in FIGS. 4 to 6.

Referring now to FIG. 4, shown therein is an example of a dialog window 400 for linking a camera to a viewport in the layout and in which a user can specify the associated camera, its floor and room number (along with a phone number for the room, if applicable), dispatch phone information, risk factors, and patient information such as, but not limited to, their patient name and patient age. Data obtained from or derived by the dialog window 400 may be stored for future auditing.

Referring now to FIG. 5, shown therein is an example of a camera settings window 500 specifying the associated camera, its IP address, port connection, camera type or encoding type, resolution, and login credentials (e.g., user name and password) for being able to access the video data provided by the associated camera.

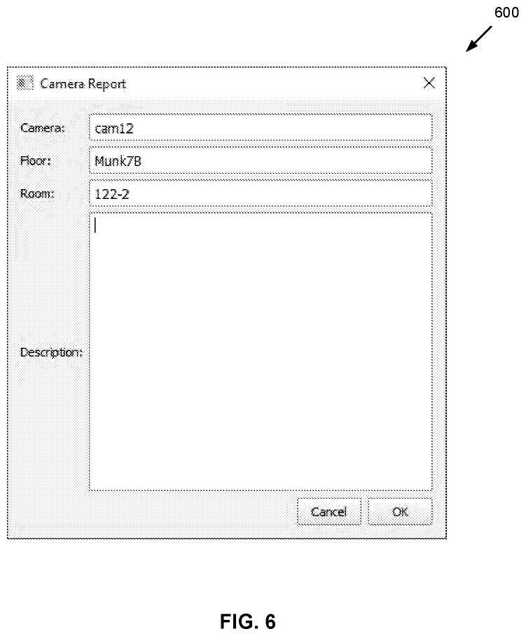

The camera settings window 500 may be provided on the RPM client 20. Referring now to FIG. 6, shown therein is an example of a logging window 600 for users to log problems associated with a given camera and site. The logging window 600 has fields for the camera identifier, its floor and room numbers, and a description for the report. The logging window 600 may be provided on the RPM client 20.

In at least one embodiment, video from the cameras 14b is streamed to the client application via real-time streaming protocol (RTSP). Each viewport of the layout in the client application is able to display one video stream. Camera controls for pan, tilt, and zoom are mapped to keyboard short-cuts.

The resolution of the camera 14b may be checked at time of connection, and an appropriate data buffer is allocated accordingly to store and display the streamed video frames from the camera 14b. Resolution changes on the camera 14b are checked and matched during receiving of video frames. A mismatch initiates a reconnection and re-allocation of the data buffer to match incoming video.

In at least one embodiment, digital zoom functionalities may be provided at a scale of 1×-5× at 0.5× steps using nearest neighbor or bilinear interpolation. Each zoom factor may have the same aspect ratio.

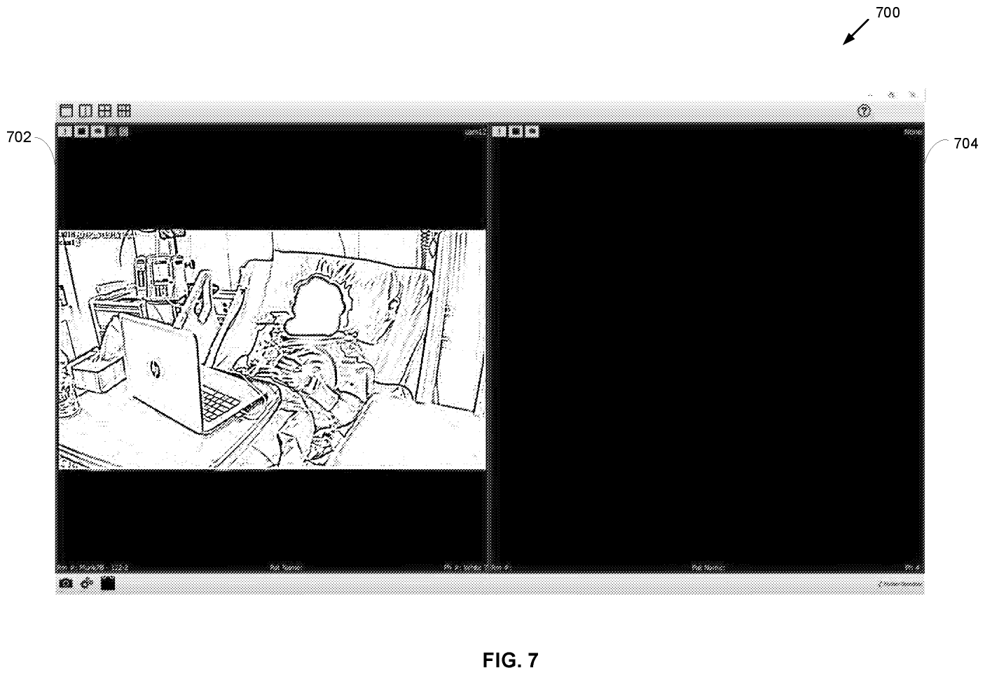

Referring now to FIG. 7, shown therein is an example of motion highlight overlay 700 on a video stream in which detected motion is highlighted with an outline in a given color, such as red. In at least one embodiment, different colors or different thicknesses of the outline may be used to indicate different types of movements such as a risky movement (i.e. the patient is getting closer to the edge of the bed and may fall out of the bed or the patient is having seizures). In at least one of these embodiments, identifying different types of movements is possible when the characteristics of the movement are defined and the edges of the bed and/or person are delineated. Further, identifying different types of movements may be possible using edges or through bounding boxes from classifier output (e.g., with a machine learning approach); bounding boxes overlapping (e.g., person+bed) may provide certain indications of risky movement. In the left window pane 702, a video stream from a camera 14b is received by the client application. In the right window pane 704, there is no video being streamed.

In at least one embodiment, the video stream from the camera 14b is processed using different motion detection techniques. Motion areas are identified by determining the foreground/background pixels using a mixture of Gaussian models. The foreground image captures the patient motion which is then used to create a contour. For example, the foreground image is then processed to remove noise using median blur filtering, and a threshold is applied to generate a binary image that corresponds to areas where motion occurs. The outlines of the areas are extracted, and the size of each area is checked against an area threshold to filter out small regions which may be due to noise or small motion. The outlines are overlaid onto the (non-processed) second video frame to highlight the motion areas. The area threshold can be changed in the client application by users to set the sensitivity of motion highlighting according to the context of the scene (e.g., depending on how important it is to monitor small movements for a given patient). For example, some patients may be more at risk of having seizures, and the sensitivity for motion highlighting may be increased in such cases to detect small motions. In contrast, some patients are not at risk of seizures but are at risk of falling out of bed, and the sensitivity for motion highlighting can be reduced in such cases to ignore small motions and only detect larger motions. This feature is used to help observers identify patient movements. The motion detection feature can be turned on and off by the observer depending on the patient that is being remotely viewed.

In at least one implementation of the motion detection techniques: both the first video frame and second video frame include the patient; the motion detection technique subtracts the first video frame from the second video frame to get the difference image; the difference image captures the patient motion, which is then used to create a contour; and when a third video frame is available, the second video frame is subtracted from the third video frame to get a new difference image to create a new contour. The motion detection technique may be further based on machine learning, in which, for example, motion analysis is based on contour movement data, such as the coordinates of the contours at different times or data derived from the coordinates (e.g., vectors, gradients, or partial derivatives).

In at least one embodiment, the server 12 and RPM client 20 may also have artificial intelligence (AI) based motion detection capabilities which are provided by models and predictive engines stored at the server 12. The aforementioned image-based motion detection employs image analysis techniques for simple motion detection and it may not differentiate if the motion is coming from the patient or what is the patient's intention for the motion. AI-based motion detection combines object detection with motion analysis. Trained models for object detection may be used as single-shot multi-box detectors to delineate object regions of interest (ROIs) in incoming video frames. This allows the RPM client 20 to only analyze motion within a patient bounding box. Certain types of detected motion within the patient bounding box may then trigger audio and visual alerts, improving true positives and reducing false negatives. Alternatively, or in addition thereto, the RPM client 20 can run part or all of the programming of the AI-based motion detection. The AI-based motion detection may be implemented by the predictive engine 132 using one or more machine learning models.

In at least one embodiment, the RPM client 20 also has gaze-tracking capabilities. A front-facing camera is mounted at the workstation for the RPM client 20 to detect the movement of the head and eyes of the observer. The front-facing camera provides observer video data which is a series of observer video frames. Faces may be detected using a Haar cascade classifier algorithm on pre-trained data, and eyes may be detected and tracked by application of circular Hough transform and constrained local models (CLM). A calibration routine with known screen points coupled with detected face and eye positions may be used to map the face and eye orientation to screen coordinates. The detected face of the observer and the centroids of the detected eye locations may be calculated from each observer video frame and used to interpolate shifts with respect to screen coordinates. If the observer is not engaged in another action through the client application and is focused on some object that is off screen from the monitor of the RPM client 20, then an audio alert may be played to notify the observer to move their eyes to watch the viewports shown on the monitor of the RPM client 20.

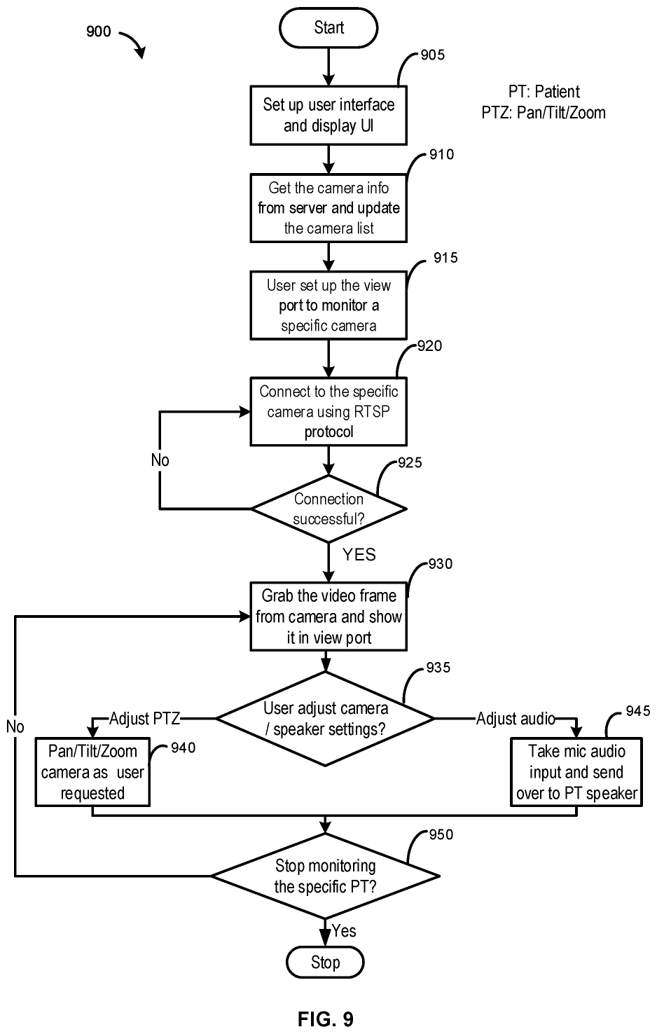

Referring now to FIG. 9, shown therein is a flow chart of an example embodiment of a method 900 of controlling a camera which may be used in at least one embodiment of the remote patient monitoring system 10 of FIG. 1A.

At act 905, the RPM client 20 starts to set up a user interface and display the user interface.

At act 910, the RPM client 20 gets an updated available camera list and information on each camera 14b from the server 12. The information about the cameras 14b may be obtained from the networked monitoring devices 14a.

At act 915, the RPM client 20 sets up a viewport to receive video from a specific camera 14b to monitor a specific patient.

At act 920, the RPM client 20 connects to the camera 14b using RTSP protocol.

At act 925, the RPM client 20 determines whether the connection was successful. If the connection is not successful, the method 900 returns to act 920 where connection is attempted again. If the connection is successful, then the method 900 proceeds to act 930.

At act 930, the RPM client 20 receives a video frame from the camera 14b and displays it in the viewport.

At act 935, the RPM client 20 determines whether an observer is sending a request to adjust the camera or the speaker setting. If a camera adjustment (e.g., adjust PTZ) is requested, the method 900 proceeds to act 940. If an audio adjustment is requested, the method proceeds to act 945.

At act 940, the RPM client 20 provides pan, tilt, and/or zoom control messages to the camera 14b according to the request from the observer. The method 900 then proceeds to act 950.

Alternatively, at act 945, the RPM client 20 receives microphone audio input data from the observer and sends it to the patient speaker according to a request from the observer. The method 900 then proceeds to act 950.

At act 950, the RPM client 20 checks whether the observer has submitted a command to stop using a particular camera 14b (or a particular networked monitoring device 14a on subnet 14) to monitor a given patient. If a stop monitoring command is not received, the method 900 returns to act 930. If a stop monitoring command is received, the method 900 ends.

In at least one embodiment, the RPM client 20 has two-way audio capabilities. A headset with a built-in microphone and audio output is worn by the observer to communicate to the patient or in-room staff through the client application. Audio from the patient room is collected from the cameras 14b and is streamed via RTSP to the client application (directly or through the server 12) along with video data. Audio data from the patient room is provided to an audio system on the RPM client 20 for output to the observer. Only audio from a currently active camera 14b is provided to the audio system. Audio to the camera 14b makes use of the camera's audio back channel. Audio data begins to be collected from the input device (e.g., microphone on headset) upon the observer pressing a mapped keyboard short-cut or other input element. Upon release of the dedicated short-cut, audio data collection halts. Audio data is sampled (e.g., at a 16-bit and 8 kHz sampling rate) and encoded with, for example, an audio pulse-code modulation (PCM) codec.

In at least one embodiment, the RPM client 20 has touch-to-call capabilities. The observer can associate a camera 14b with a patient, a nurse, and the nurse dispatch phone in the client application. In case of an event that requires immediate action from the nurse, the observer can make a dispatch call with the “Touch to Call” icon on the client application to send a call to the nurse. In such cases, a request is sent from the client application to a cloud phone service provider via its REST API. The cloud phone service provider starts a call to the nurse's cell phone/hospital phone and waits for a response from the nurse. It then notifies server 12 that the nurse responds to the call via the touch-to-call notification REST API. The server 12 then notifies the client application if the nurse has responded using the WebSocket protocol.

In at least one embodiment, the server 12 has two-way audio real-time translation capabilities. Built on advanced machine learning based speech recognition, speech synthesis, and natural language processing (NLP), the two-way audio translation allows the observers and patients being monitored to communicate in their native languages. The audio from one party is first transcribed to text, the text is then translated into the other party's preferred language, and then the translated text is then converted to speech. This allows patients who are not English speakers to communicate with the observer easily without the help of a translator.

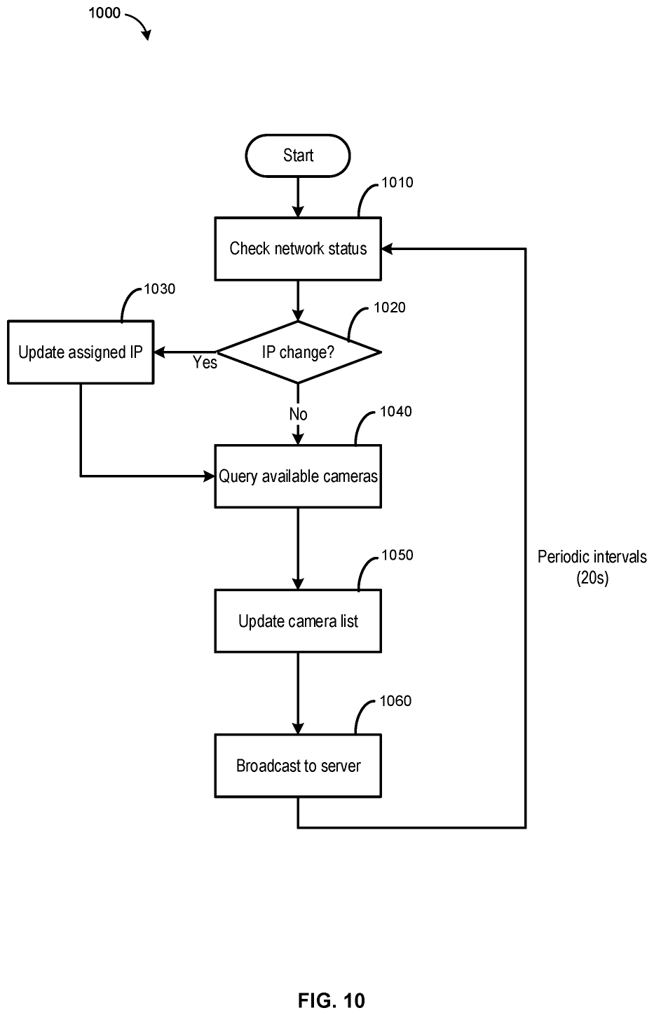

Referring now to FIG. 10, shown therein is a flow chart of an example embodiment of a method of managing available/monitoring cameras 1000 which may be used in at least one embodiment of the remote patient monitoring system 10 of FIG. 1A.

At act 1010, a networked monitoring device 14a checks the network status. In particular, the networked monitoring device 14a may check whether the connectivity is good or meets a particular threshold (e.g., below a preset error rate). Alternatively, or in addition, the networked monitoring device 14a may check for any IP address changes. The networked monitoring device 14a is a device that is deployed to a particular subnet 14. For example, the networked monitoring device 14a may be plugged into an available jack at a nursing station. In different embodiments, the networked monitoring device 14a may cover a portion of a floor, an entire floor, portions of multiple floors, and/or multiple entire floors.

At act 1020, the networked monitoring device 14a determines whether its own IP has changed. If there is an IP change, the method 1000 proceeds to act 1030. If there is not an IP change, the method 1000 proceeds to act 1040.

At act 1030, the networked monitoring device 14a updates the assigned IP address.

At act 1040, the networked monitoring device 14a queries the available cameras 14b that are connected on the same subnet 14.

At act 1050, the networked monitoring device 14a updates the camera list to include only the cameras 14b that are available for sending video data to the client application.

At act 1060, the networked monitoring device 14a broadcasts the camera list to the server 12. The method 1000 may then return to act 1010 after a preset time (e.g., 20 seconds).

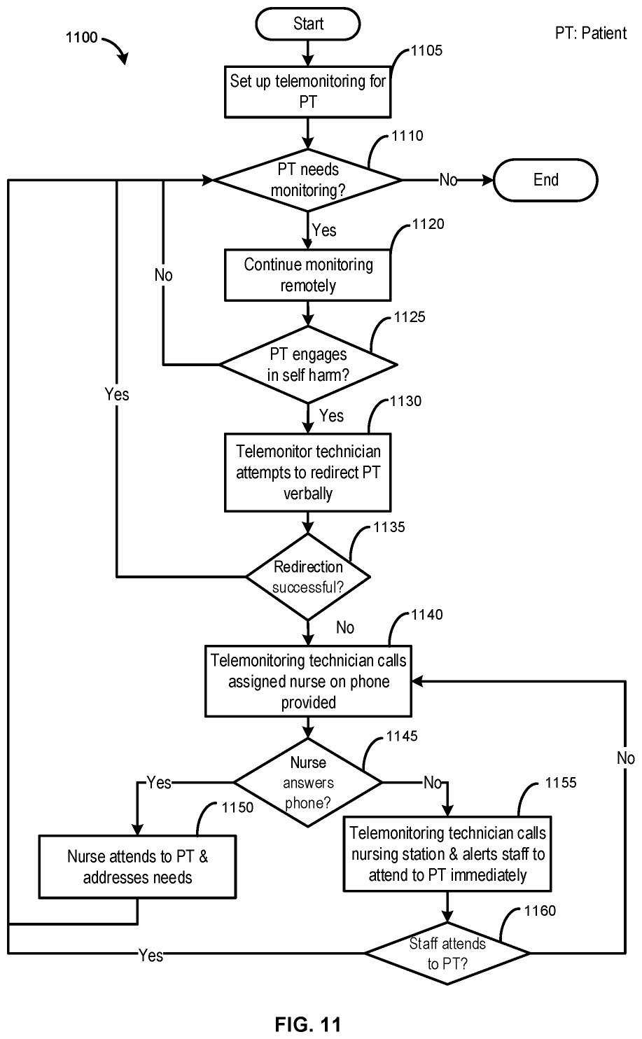

Referring now to FIG. 11, shown therein is a flow chart of an example embodiment of a method 1100 of automating a self-harm response which may be used in at least one embodiment of the remote patient monitoring system 10 of FIG. 1A.

At act 1105, the RPM client 20 starts to set up tele-monitoring for a specific patient.

At act 1110, an observer (e.g., a tele-monitor) determines whether the patient needs continued remote monitoring. The determination may be input (e.g., by keyboard, audio command, gesture received by webcam) into the RPM client 20, and the RPM client 20 may send the input to the server 12. Alternatively, or in addition, the RPM client 20 may make the determination for the observer based on, for example, input from the camera 14b. If the patient does not need remote monitoring (conditions deteriorated or conditions improved), the remote monitoring stops; if the patient still needs remote monitoring, the remote monitoring continues.

At act 1120, the RPM client 20 continues to provide video and/or audio to an observer to be able to monitor the patient.

At act 1125, the observer determines whether the patient is engaging in self harm. The determination may be input (e.g., by keyboard, audio command, gesture received by webcam) into the RPM client 20, and the RPM client 20 may send the input to the server 12. Alternatively, or in addition, the RPM client 20 may make the determination for the observer based on, for example, input from the camera 14b. If this condition is not true (i.e., the patient is not engaging in self harm), then the method 1100 returns to act 1110. If this condition is true (i.e., the patient is engaging in self harm), then the method 1100 proceeds to act 1130.

At act 1130, the observer attempts to redirect the patient verbally. During act 1130, the RPM client 20 can receive a control input from the client application to send audio data to the patient room. The observer can then speak into a microphone to provide the audio data. The RPM client 20 receives the audio data, and sends the audio data to the patient room for broadcast to the patient through a speaker in the patient's room.

At act 1135, the observer determines whether the redirection was successful. The determination may be input (e.g., by keyboard, audio command, gesture received by webcam) into the RPM client 20, and the RPM client 20 may send the input to the server 12. Alternatively, or in addition, the RPM client 20 may make the determination for the observer based on, for example, input from the camera 14b. If the redirection is successful, the method 1100 then returns to act 1110. If the redirection is not successful, the method 1100 proceeds to act 1140.

At act 1140, the observer calls an assigned nurse. During act 1140, the RPM client 20 may receive a control input from the observer to contact the assigned nurse, and the RPM client 20 may send an electronic message or attempt to initiate a voice call with the nurse's phone using the touch-to-call feature.

At act 1145, the observer determines whether the assigned nurse answers the phone. The determination may be input (e.g., by keyboard, audio command, gesture received by webcam) into the RPM client 20, and the RPM client 20 may send the input to the server 12. Alternatively, or in addition, the server 12 may make the determination for the observer based on, for example, network data representing connection with the nurse's phone. If the nurse's phone is answered, the method 1100 proceeds to act 1150. If the nurse's phone is not answered, the method 1000 proceeds to act 1155.

At act 1150, the assigned nurse attends to the patient and addresses the patient's needs. The nurse's actions may be input (e.g., by keyboard, audio command, gesture received by webcam) into the RPM client 20, and the RPM client 20 may send the input to the server 12. Alternatively, or in addition, the RPM client 20 may signal to the server 12 the nurse's actions based on, for example, input from the camera 14b. The method 1100 then returns to act 1110.

Alternatively, at act 1155, the observer calls a nursing station and alerts the nursing staff to attend to the patient immediately. During act 1155, the RPM client 20 may receive a control input from the client application to send an electronic message or initiate a voice call with the nursing station.

At act 1160, the observer determines whether the nursing staff is attending to the patient. The determination may be input (e.g., by keyboard, audio command, gesture received by webcam) into the RPM client 20, and the RPM client 20 may send the input to the server 12. Alternatively, or in addition, the RPM client 20 may make the determination for the observer based on, for example, input from the camera 14b. If this condition is true (i.e., the nursing staff is attending to the patient), the method 1100 returns to act 1110. If this condition is not true (i.e., the nursing staff is not attending to the patient), the method 1100 returns to act 1140 (e.g., for further attempts to contact the nurse).

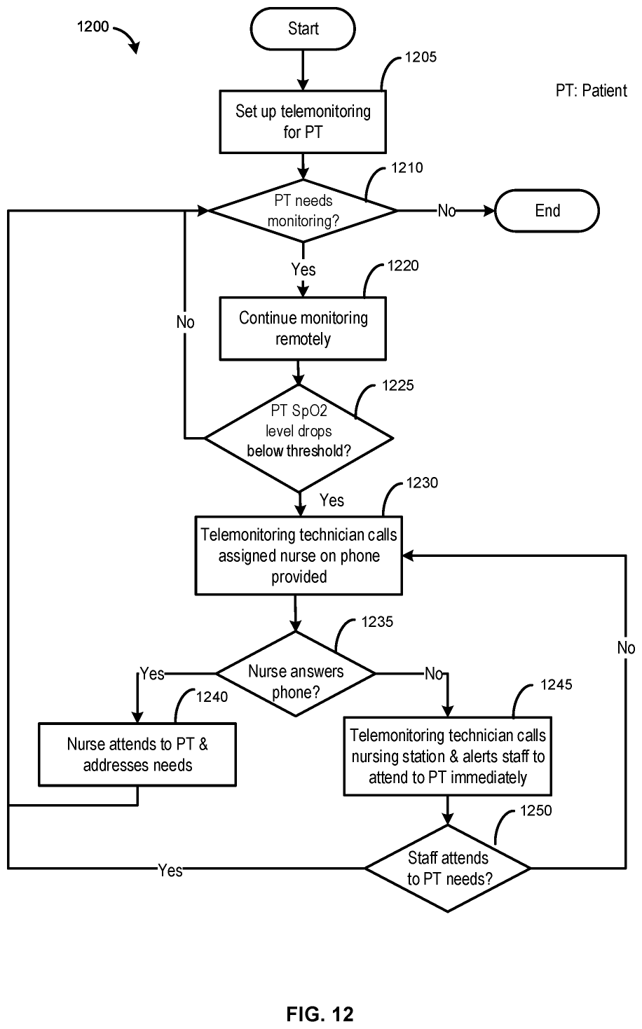

Referring now to FIG. 12, shown therein is a flow chart of an example embodiment of a method 1200 of automating an oxygen saturation abnormality response which may be used in at least one embodiment of the remote monitoring system 10 of FIG. 1A.

At act 1205, the RPM client 20 starts to set up for tele-monitoring of a specific patient.

At act 1210, an observer (e.g., a tele-monitor) determines whether the patient needs continued remote monitoring. The determination may be input (e.g., by keyboard, audio command, gesture received by webcam) into the RPM client 20, and the RPM client 20 may send the input to the server 12. Alternatively, or in addition, the RPM client 20 may make the determination for the observer based on, for example, input from the camera 14b. If the patient does not need remote monitoring (conditions deteriorated or conditions improved), the remote monitoring stops; if the patient still needs remote monitoring, the remote monitoring continues.

At act 1220, the RPM client 20 continues to provide video and/or audio to the observer to be able to monitor the patient.

At act 1225, the RPM client 20 obtains the patient's SpO₂ data from the physiological data that is received and determines whether the patient's SpO₂ level drops below an SpO₂ threshold. If this condition is not true (i.e., the patient's SpO₂ level does not drop below an SpO₂ threshold), the method 1200 returns to act 1210. If this condition is true (i.e., the patient's SpO₂ level drops below an SpO₂ threshold), the method 1200 proceeds to act 1230.

At act 1230, the observer directs the RPM client 20 to call an assigned nurse. During act 1230, the RPM client 20 may receive a control input from the client application, provided by the observer, to contact the assigned nurse, and the server 12 may send an electronic message or attempt to initiate a voice call with the nurse's phone.

At act 1235, the observer determines whether the assigned nurse answers the phone. The determination may be input (e.g., by keyboard, audio command, gesture received by webcam) into the RPM client 20, and the RPM client 20 may send the input to the server 12. Alternatively, or in addition, the server 12 may make the determination for the observer based on, for example, network data representing connection with the nurse's phone. If the nurse's phone is answered, the method 1200 proceeds to act 1240. If the nurse's phone is not answered, the method 1200 proceeds to act 1245.

At act 1240, if the nurse's phone is answered, the observer uses the RPM client 20 to send an electronic message or audio data from the observer received from the client application to the assigned nurse to instruct the assigned nurse to attend to the patient and address the patient's needs. The method 1200 then returns to act 1210.

At act 1245, if the nurse's phone is not answered, the observer calls a nursing station and alerts the nursing staff to attend to the patient immediately. During act 1245, the RPM client 20 may receive a control input from the client application to send an electronic message or initiate a voice call with the nursing station.

At act 1250, the observer determines whether the nursing staff is attending to the patient. The determination may be input (e.g., by keyboard, audio command, gesture received by webcam) into the RPM client 20, and the RPM client 20 may send the input to the server 12. Alternatively, or in addition, the RPM client 20 may make the determination for the observer based on, for example, input from the camera 14b. This may be determined if the RPM client 20 receives a message from the nurse station indicating that the nursing staff is attending to the patient. Alternatively, the RPM client 20 may receive an input from the client application to indicate that the nursing staff is attending to the patient. This may be provided by the observer if they see in the video data that the nursing staff is attending to the patient. If the condition at act 1250 is true (i.e., the nursing staff is attending to the patient), the method 1200 returns to act 1210. If the condition at act 1250 is not true (i.e., the nursing staff is not attending to the patient), the method 1200 returns to act 1230.

In at least one embodiment, the system 10 has continuous SpO₂ monitoring capabilities. Continuous SpO₂ monitoring may be accomplished by streaming data from an FDA approved pulse oximeter device over Bluetooth, for example. For a certain patient population such as lung transplant patients, it has been shown that using continuous SpO₂ monitoring can result in more timely response to patient health deterioration. The continuous SpO₂ monitoring can be implemented with one of two (or both) approaches. If the hospital has continuous SpO₂ data available in the EPR system, then an HL7 (or FHIR) feed is connected to the RPM client 20 to show the patient SpO₂ data on the camera view in real time. If the SpO₂ data is not available in an electronic patient record (EPR), a Bluetooth SpO₂ monitor is used and the data is sent to an embedded computing device, and the SpO₂ data is relayed from the embedded computing device to the RPM client 20 using WebSocket.

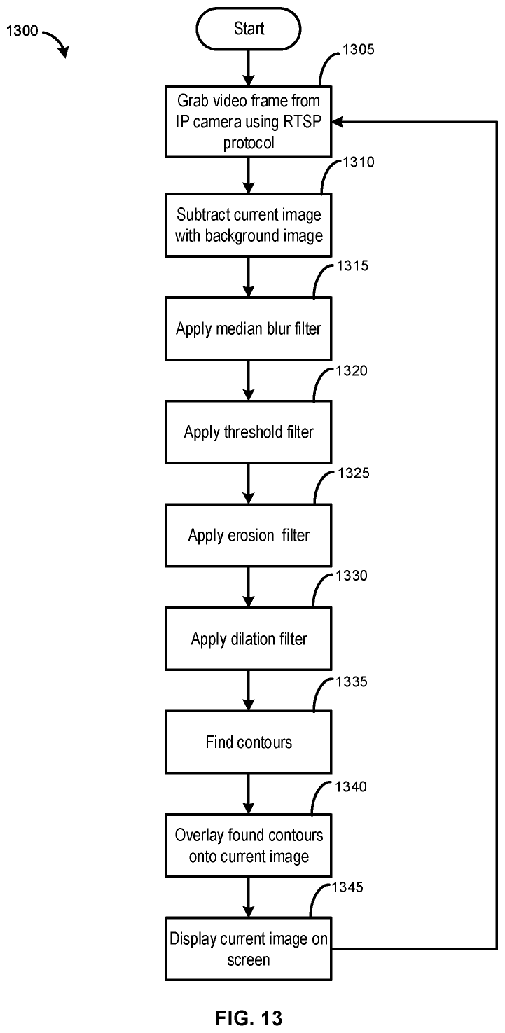

Referring now to FIG. 13, shown therein is a flow chart of an example embodiment of a method 1300 of detecting and displaying patient motion which may be used in at least one embodiment of the remote monitoring system 10 of FIG. 1A.

At act 1305, the RPM client 20 receives a video frame from a camera 14b using the RTSP protocol. The received video frame is referred to as a current image. A background image is saved from a previous video frame in which the patient is not present (or, for example, a previous picture taken of the bed without the patient on it).

The RPM client 20 may define a reference background image including the patient from the video frames and defining the current image with the patient. The reference background image and current image are each made up of their respective image pixels.

At act 1310, the RPM client 20 creates a background model with the background image pixels by fitting the pixel values to a mixture of Gaussian distributions, each with independent weight, mean and variance. The background model of Gaussian distributions is used for classifying any new incoming pixels by comparing the Mahalanobis distance to the center of the distributions (e.g., how many standard deviations away it is from the mean). The RPM client 20 determines if the pixels in the current image are part of the background or the foreground by calculating how close they are from the background model via Mahalanobis distance. If they are close to the model, they are classified as background pixels. If not, they are classified as foreground pixels. The RPM client 20 then collects the current image pixels classified as foreground pixels to generate a foreground image.

As an example, suppose two Gaussian distributions are used to create the background model and for each Gaussian distribution it can be defined using a mean and a variance. Next, each distribution is assigned a weight and then added up. The following formula may be used:

Model=weight1*Gaussian(mean,variance)+weight2*Gaussian(mean,variance)

The model is a mixture of both Gaussian distributions with its respective weight. In order to determine the weight, mean and variance, the background image pixels are used to fit this model to derive their values. Once the weight, mean and variance are known, they can be plugged into the equation to test any new incoming pixel to see if it is close to this model (e.g., if its value within 3 standard deviations of the mean of the model).

By way of example, suppose an incoming pixel has an intensity of 200. Suppose further that the Gaussian model provides that the background pixel values are in the range of [50, 150] with a mean of 100. The probability that the incoming pixel is a background pixel is therefore low, so it can be classified as a foreground pixel.

The Mahalanobis distance is a mathematical way to calculate the distance like the simple distance of 200−100=100, so it can be determined whether the pixel value is close to the mean value of the model distribution. The Mahalanobis distance accounts for the covariance and spread of the background model. Different Mahalanobis distances essentially correspond to different sized ellipsoids centered around the spread of the background model pixels, where each distance value is an ellipsoidal decision boundary corresponding to a standard deviation away from the mean.

The RPM client 20 may then apply one or more filters to the foreground image. For example, at act 1315, the RPM client 20 may apply a median blur filter to the foreground image to obtain a first filtered foreground image. As another example, at act 1320, the RPM client 20 may then apply a subsequent threshold filter to the first filtered foreground image to obtain a second filtered foreground binary image—labelled as a “binary” image because it contains only ‘0’ and ‘1’ values. As another example, at act 1325, the RPM client 20 may apply a further subsequent filter such as an erosion filter to the second filtered foreground binary image to obtain a third filtered foreground binary image. As another example, at act 1330, the RPM client 20 may apply a further subsequent filter such as a dilation filter to the third filtered foreground binary image to obtain a fourth filtered foreground binary image.

At act 1335, the RPM client 20 then identifies contours in the fourth filtered foreground binary image by finding borders of ‘1’ regions. There may be multiple ‘1’ regions, so multiple contours may be identified. This may be done by applying an appropriate edge detection technique to the fourth filtered foreground binary image. The RPM client 20 may find contours that have areas larger than a predefined sensitivity value.

At act 1340, the RPM client 20 overlays the contours onto the current image.

At act 1345, the RPM client 20 displays the overlaid current image (with the contours) on the RPM client 20. The method 1300 returns to act 1305 for processing the next video frame in a similar manner.

In at least one embodiment, the foreground images (or filtered foreground images) may be masked images (or filtered masked images).

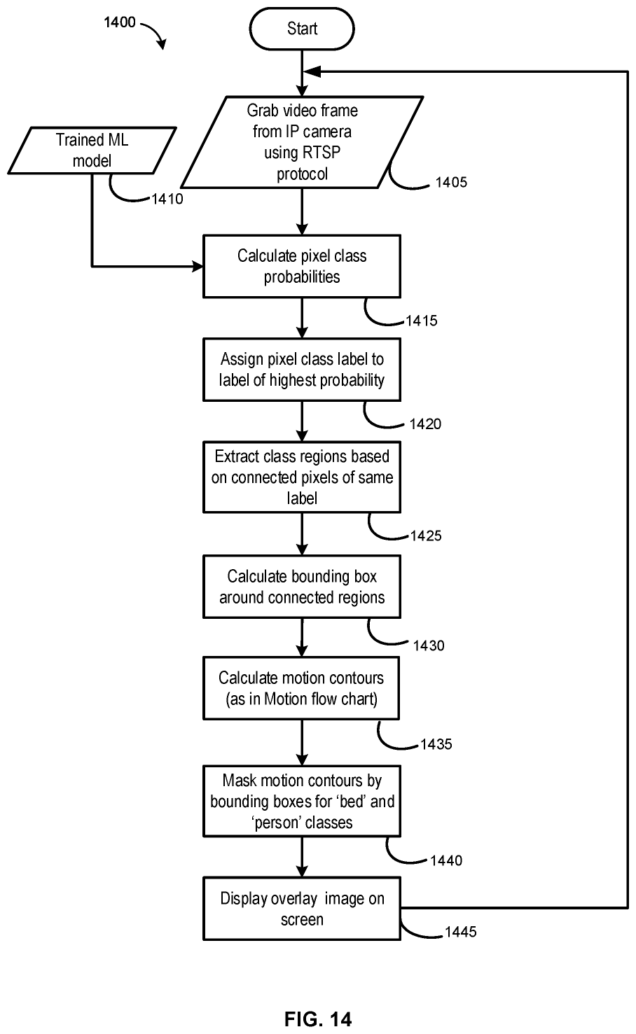

Referring now to FIG. 14, shown therein is a flow chart of an example embodiment of a method 1400 of applying machine learning to detecting and displaying patient motion which may be used in at least one embodiment of the remote monitoring system 10 of FIG. 1A.

At act 1405, the RPM client 20 receives a video frame from a camera 14b using the RTSP protocol. The video frame includes an array of pixels which can be grouped into regions in the later acts of method 1400.

At act 1410, the RPM client 20 selects a trained machine learning (ML) model from the machine learning models 126. The trained ML model can be internally generated (e.g., during execution of method 1400) or externally produced (e.g., from supervised ML using previously obtained video data).

At act 1415, the RPM client 20 calculates pixel class probabilities from the video frame using the trained ML model. The pixel class probabilities estimate the probability that a given pixel belongs to a certain class which represents a certain type of object in the video frame. Different classes may include the physical objects (e.g., bed, person, chair) in the video frame.

At act 1420, the RPM client 20 assigns a pixel class label to a given pixel that is associated with the largest pixel class probability determined at act 1410.

At act 1425, the RPM client 20 extracts class regions based on connected pixels having the same pixel class label.

At act 1430, the RPM client 20 calculates a bounding box around the connected regions. The connected regions are the class regions that have connected pixels having the same class type. The RPM client 20 may calculate multiple bounding boxes, and the bounding boxes may be used to filter motion regions and trigger alarms (e.g., a patient moving around or getting out of a bed or chair).

At act 1435, the RPM client 20 calculates motion contours from the video frame. This can be done in a manner similar to that of method 1300. For example, calculating motion contours can include one or more of creating difference images, applying filters, finding contours, and overlaying contours to an image that includes the class regions determined at act 1430. The RPM client 20 may find motion contours that have areas larger than a predefined sensitivity value.

At act 1440, the RPM client 20 generates masks for the motion contours by bounding boxes for the “bed” and “person” classes. The bed and person classes can be, for example, generated by the selected trained ML model or obtained from an external source. Obtaining masks for the motion contours in this manner generates the contour data for an overlay image showing motion around the person.

At act 1445, the RPM client 20 displays the overlay image on the RPM client application viewport. The method 1400 returns to act 1405 for processing the next video frame in a similar manner.

Method 1400 advantageously uses classified regions to filter patient and bed motion areas. The use of classified regions improves the functioning of the system 10 by allowing it to more simply, filter motion regions based on detected object. Advantageously, the machine learning approach enables creation of regions of interest (ROIs) over detected objects (e.g., object bounding boxes), which enables the filtering of normal motion detection according to the ROIs that are relevant (e.g., bed or person).

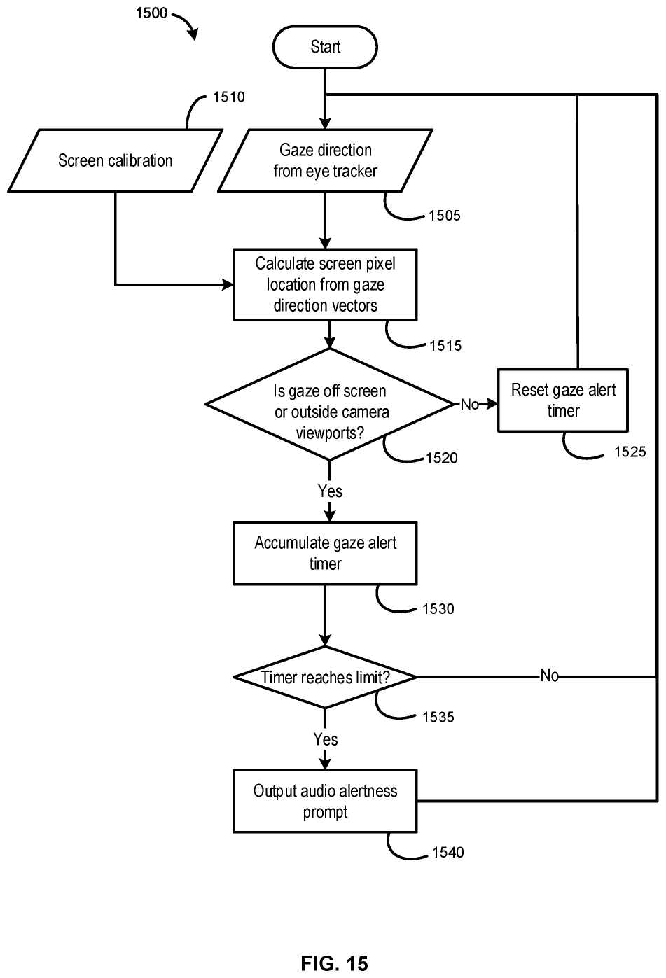

Referring now to FIG. 15, shown therein is a flow chart of an example embodiment of a method 1500 of gaze alertness tracking which may be used in at least one embodiment of the remote monitoring system 10 of FIG. 1A.

At act 1505, the RPM client 20 receives image data on the gaze direction of the observer (e.g., a tele-monitor) from an eye tracker.

At act 1510, the RPM client 20 performs screen calibration of its screen or screens. The RPM client 20 may then send calibration data on the screen calibration to the RPM client 20. Calibration is performed through the display of known image centers (pixel location) on the screen while the observer under eye tracking, directs the gaze at the image centers. This computes the mapping between detected pupil center, direction, and pixel location for the extent of the screen.

At act 1515, the RPM client 20 calculates a screen pixel location from gaze direction vectors obtained from an eye tracker. The gaze direction vectors represent the direction in which the observer is looking at the point at which the vectors interest the display is determined to obtain the screen pixel location of the observer's gaze.

At act 1520, the RPM client 20 identifies if the gaze direction is off screen (or outside of any of the camera viewports). If this condition is not true (i.e., the gaze direction is not off screen), the method 1500 proceeds to act 1525. If the condition at act 1520 is true (i.e., the gaze direction is off screen), the method 1500 proceeds to act 1530.

If the observer is properly viewing the display, then at act 1525, the RPM client 20 resets the gaze alert timer. The method 1500 returns to act 1505. The gaze alert time represents the amount of time that the observer is not looking at the display.

If the observer is not properly viewing the display, then at act 1530, the RPM client 20 accumulates (i.e., starts incrementing) the gaze alert timer to monitor how long it has been since the observer has stopped looking at the display showing the viewport(s) of the RPM client 20.

At act 1535, the RPM client 20 identifies if the gaze alert timer reaches a limit. If this condition is not true (i.e., the gaze alert timer does not reach the limit), the method 1500 returns to act 1505. If the condition at act 1535 is true (i.e., the gaze alert timer reaches the limit), then the method 1500 proceeds to act 1540.

At act 1540, the RPM client 20 outputs an audio alertness prompt. Alternatively, or in addition, the RPM client 20 flashes the screen, generates a vibration, or otherwise alerts the observer (e.g., sends an SMS message to the observer's personal device) to indicate that the observer needs to start observing the viewport(s) again.

Method 1500 advantageously combines eye tracking with attention regions to ensure that an observer is performing remote observation of patients and triggering one or more alertness alarms when the observer is not viewing the viewport(s). This combination of gaze tracking and alerts for observing viewports associated with remote patient observation makes the RPM client 20 an integral, interactive component of the system 10 by ensuring full engagement of the observer (e.g., tele-monitor).

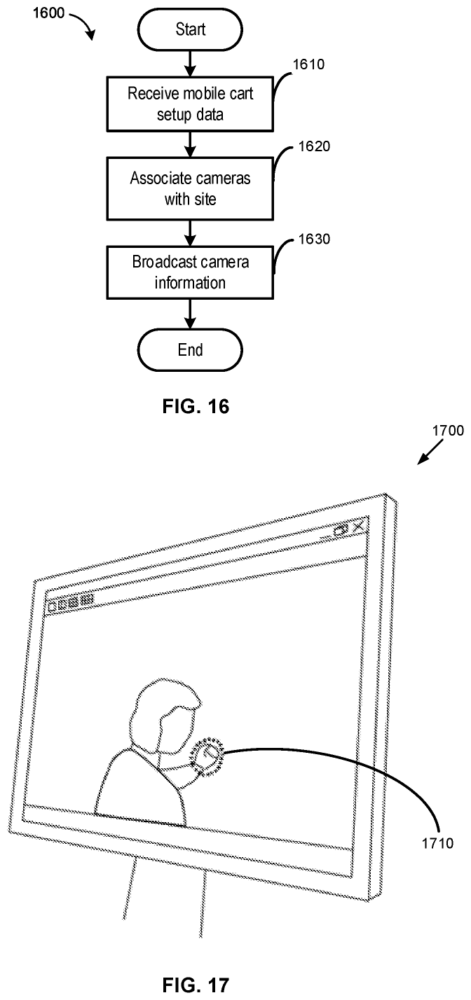

Referring now to FIG. 16, shown therein is a flow chart of an example embodiment of a method 1600 of mobile cart configuration which may be used in at least one embodiment of the remote patient monitoring system 10 of FIG. 1A.

At act 1610, the networked monitoring device 14a receives setup data from a mobile cart carrying a camera 14b.

At act 1620, the networked monitoring device 14a associates the camera 14b with a particular site. The site may be entered at the mobile cart or previously designated on a physical and/or logical map. The site can be a certain room on a certain floor of a certain building.

At act 1630, the networked monitoring device 14a broadcasts data about the camera 14b. The broadcast data can include location data such as the physical location, floor, site, GPS coordinates, or map coordinates of the camera 14b.

In at least one embodiment, the method 1600 can be used to configure the locations, identities, and/or login information of a plurality of cameras 14b.

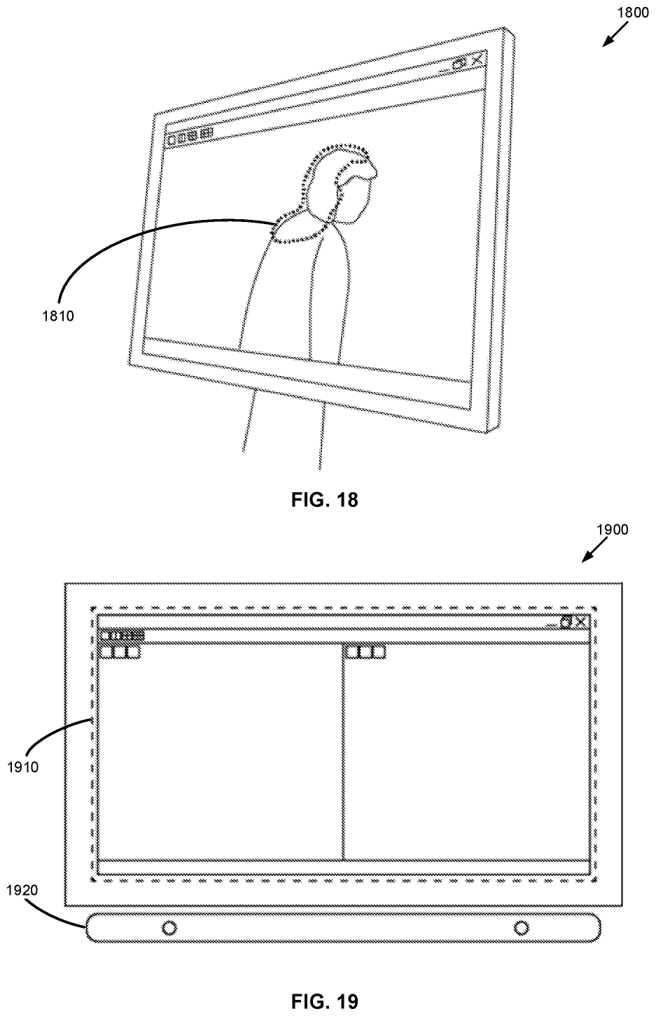

Referring now to FIGS. 17 and 18, shown therein are example screen views on an RPM client 20 that demonstrate motion detection. FIG. 17 shows an illustration of an actual example screen view of motion highlighting for a “hand raising” action 1700, where the hand tracking 1710 is shown as a dotted region. FIG. 18 shows an illustration of an actual example screen view of motion highlighting for a “getting up” action 1800, where the head and back tracking 1810 is shown as a dotted region. In FIGS. 17 and 18, the RPM client 20 controls the camera 14b to observe a seated user at a 1 meter distance.

The RPM client 20 provides motion detection and visual cues via highlighting that can be toggled on and off. For each live camera feed, a Gaussian mixture-based background/foreground segmentation is used to determine the background from foreground pixels. When turned on, the first video frame received is used as a reference background frame, where background pixels are modelled using a mixture of Gaussians. New received pixels are calculated against the background model via Mahalanobis distance to determine if they are background/foreground pixels. The Mahalanobis distance captures the covariance and spread of red, green, and blue (RGB) pixels corresponding to a background. The decision boundary corresponding to the distance is an ellipsoid, which may better capture the spread of data as compared to the sphere as with Euclidean distance. Pixels that pass as foreground via distance check and are connected together are deterministic of the motion region. In the RPM client 20, a sensitivity value corresponding to an area threshold can be adjusted, highlighting connected foreground regions only if the connected pixel area is greater than the sensitivity value.

During testing, the RPM client 20 was set up to test the performance of detecting a user's motion using one camera unit. The camera unit was positioned at a one meter distance from the user being monitored. The user sat in a chair and performed three motions repeatedly: (1) getting up and sitting down, (2) raising and lowering an arm, and (3) changing sitting position. Each motion was performed 10 times. The RPM client motion detection mode was turned on to detect these motions. A webcam was placed with its field of view over the observed user to record motions performed by the user. The recorded video was then analyzed to determine the correlation between the motions occurred in the video and the motions detected by the RPM client 20 as a highlighted visual cue via outline shown on the RPM client 20. The study was repeated with the camera unit positioned at a two meter distance from the observed user.

FIG. 17 illustrates an example of motion highlighting of the hand raising action at a 1 meter distance. FIG. 18 illustrates an example of motion highlighting of the getting up action at a 1 meter distance. The detection and correlation of the events of motion at a distance of 1 meter is listed in Table 1.

TABLE 1
Motion action and detection results at 1 meter distance

| | | Number of Detections |
|---|---|---|
| Event | Number of Occurrences | and Highlight |
| Getting Up | 10 | 10 |
| Raising Arms | 10 | 10 |
| Changing Position | 10 | 10 |

The detection and correlation of the events of motion at a distance of 2 meters is listed in Table 2.

TABLE 2
Motion action and detection results at 2 meter distance

| | | Number of Detections |
|---|---|---|
| Event | Number of Occurrences | and Highlight |
| Getting Up | 10 | 10 |
| Raising Arms | 10 | 2 |
| Changing Position | 10 | 9 |

At the one meter distance, the RPM client 20 successfully detected all three types of motions, while at the two meter distance, the RPM client 20 detected all getting up events correctly but only some of the other 2 types of motions correctly.

Referring now to FIG. 19, shown therein is an example screen view on an RPM client 20 that demonstrates eye tracking 1900. In FIG. 19, an RPM client 20 (on a laptop) is shown with a Tobii eye tracker 1920 at the bottom of the display. The RPM client 20 registers observer gaze to a screen location and invokes events based on whether the observer is looking on or off the screen. If the observer's gaze points are off screen the RPM client 20 will alert the observer that he is not looking at the screen. The gaze tracking region 1910 is shown in dashed lines at the extent of the display. The eye tracker 1920 may detect whether the gaze points are on or off screen by calling the eye tracker API. The RPM client 20 is notified by the eye tracker 1920 when the observer's gaze points are on or off screen, which informs the RPM client 20 whether the observer is looking at the screen or at something else.

During testing, a consumer-grade Tobii eye tracker (see, e.g., https://gaming.tobii.com/tobii-eye-tracker-4c/) was placed under and connected to an RPM client 20 (on a laptop). The eye tracker was calibrated to the observer and the laptop display with the calibration software provided by Tobii. The observer followed the software directions to look at the center and corners of the laptop display, and the calibration software tracked the user's gaze point by its location within the extent of the display to complete the calibration process. The eye tracking feature was implemented in the RPM client 20 to alert the user when the user's gaze was off the display. A user sitting in front of the laptop and his gaze points were tracked by the RPM client 20 (according to the method 1500 of gaze alertness tracking described above) using the eye tracker. The observed user performed two type of tasks: (1) looking at the laptop display and (2) looking away from the display. Each task was performed 11 times. A webcam was placed with its field of view over the observed user to record tasks performed by the user. The recorded video was then analyzed to determine the correlation between the tasks performed by the user in the video and the gaze on display/gaze off display events detected by the RPM client 20. Measurements were also taken to check the delay between the actual events happened (measured by the timestamp when the event happened from the recorded video) and alerts sent by the RPM client 20 (measured by the timestamp when the alerts showed up in the RPM client 20 from the recorded video).

A total of 22 gaze events were measured where the observer looked at and away from the display, with corresponding alerts in the RPM client 20 for each. The RPM client 20 outputted correct alerts corresponding to each of the 22 events as summarized in Table 3. The delay between the actual events and the alerts sent by the RPM client 20 was less than 1 second. In particular, Table 3 shows alert events from the RPM client 20, matching number of occurrences when gaze is on/off the display.

TABLE 3
Alert events from RPM client - matching number
of occurrences when gaze is on/off the display

| Event | Number of Occurrences | Number of Detections |
|---|---|---|
| Gaze On Display | 11 | 11 |
| Gaze Off Display | 11 | 11 |

Bluetooth SpO₂ streaming was also experimentally tested. A Nonin Model 3230 Bluetooth LE pulse oximeter (see, e.g., https://www.nonin.com/products/3230/) was worn on a patient's index finger to measure the patient's oxygen saturation. The pulse oximeter was paired with a small embedded computer on a mobile cart via Bluetooth connection protocol. A custom software program was implemented and run on the embedded computer to receive SpO₂ measurements and events transmitted by the pulse oximeter via wireless Bluetooth communication. The pulse oximeter transmitted the measurements (SpO₂ reading and pulse rate) once every second. The embedded computer streamed the SpO₂ measurements and events serially to the RPM client 20 via socket communication over the hospital local area network. The RPM client 20 displayed the streamed SpO₂ measurements in the monitoring window for the monitored patient onscreen. A user performed two type of tasks: (1) putting the pulse oximeter on the patient's index finger and leaving it running for 30 seconds; and (2) taking it off the patient's index finger and leaving it off for 30 seconds. The pulse oximeter had automatic on and off functionality so the user did not need to manually turn it on or off. A webcam was placed with its field of view over the pulse oximeter worn by the patient to record visible display readings for correlation with visual SpO₂ display from the RPM client 20.

For event detection and correlation, two types of events were observed: (1) pulse oximeter on/off event and (2) pulse oximeter display readings that were in sync with the SpO₂ display from the RPM client 20 for longer than 5 seconds. Table 4 summarizes detection results for pulse oximeter on/off detection correlation. It shows that the RPM client 20 successfully detected all 22 events on events and 22 off events. Table 5 shows that the readings from the RPM client 20 were successfully in sync with the readings from the pulse oximeter for all 22 observed times. Streaming measurements were also collected over a period of 12 minutes with an average delay between corresponding webcam video frame to measurement of less than 1 second with 0 missed readings. In particular, Table 4 shows detected pulse oximeter events from a Nonin 3230 Bluetooth pulse oximeter and the RPM client 20.

TABLE 4
Detected Pulse Oximeter Events from Nonin 3230
Bluetooth pulse oximeter and RPM client

| Event | Number of Occurrences | Number of Detections |
|---|---|---|
| Pulse Oximeter On | 22 | 22 |
| Pulse Oximeter Off | 22 | 22 |

Table 5 shows detected in-sync events from a Nonin 3230 Bluetooth pulse oximeter and the RPM client 20.

TABLE 5
Detected in-sync Events from Nonin 3230 and RPM client

| Event | No. of Occurrences | No. of Detections |
|---|---|---|
| Reading in sync more than 5 sec | 22 | 22 |

The systems and methods described above advantageously can be implemented using multiple RPM clients and networked monitoring devices to scale up to monitor hundreds of patients from one hospital site or multiple sites, and also to monitor patients in different hospitals or even from their home.

In at least one embodiment, the systems and methods described above advantageously can employ a mobile patient monitoring cart by incorporating a camera, speaker, and other measuring devices into one mobile unit which can be deployed to different rooms/sites with minimum setup effort (e.g., plug and play).

The systems and methods described above advantageously can be deployed in either a small clinic which consists of a single network or a large enterprise for which its network structure is often complex and restricted (e.g., both wired/wireless networks, different subnets, multiple VLANs, multiple sites, different visibility, and dynamic IP vs. static IP).

The systems and methods described above advantageously can be deployed in (or scaled up to) a complex network environment without loss of reliability. In a complex network environment, a number of issues can arise, such as: a wireless network being available in most of the patient rooms but not all; the reliability of the wireless network being poor so it is not a good option for RPM; a wired network spanning multiple hospital campuses and being divided into multiple subnets; a camera or any monitoring device plugged into the network not being easily discoverable unless it is capable to push data to a (central) server; the network being configured to use dynamic IP or static IP, and if a camera is configured to use static IP then it cannot be used in a dynamic IP network. Some or all of these issues are addressed in some or all of the embodiments described above. For example, subnets can be defined using locators to cover the different sites where possible, and for locations in which locators are not possible the smart “mobile cart” can be used and is capable of being plugged into either a dynamic or a static IP network.

The systems and methods described above advantageously can save computer resources by providing the RPM client with a multi-patient display so that an observer can observe more than one patient at a time or use fewer displays to observe multiple patients. The savings in computer resources can also be achieved by reducing the number of client applications that need to be started or managed to monitor multiple patients. Additionally, two-way audio may be enabled on the RPM client, such that the tele-monitor can communicate with multiple patients over one client application, further resulting in a savings in computer resources.

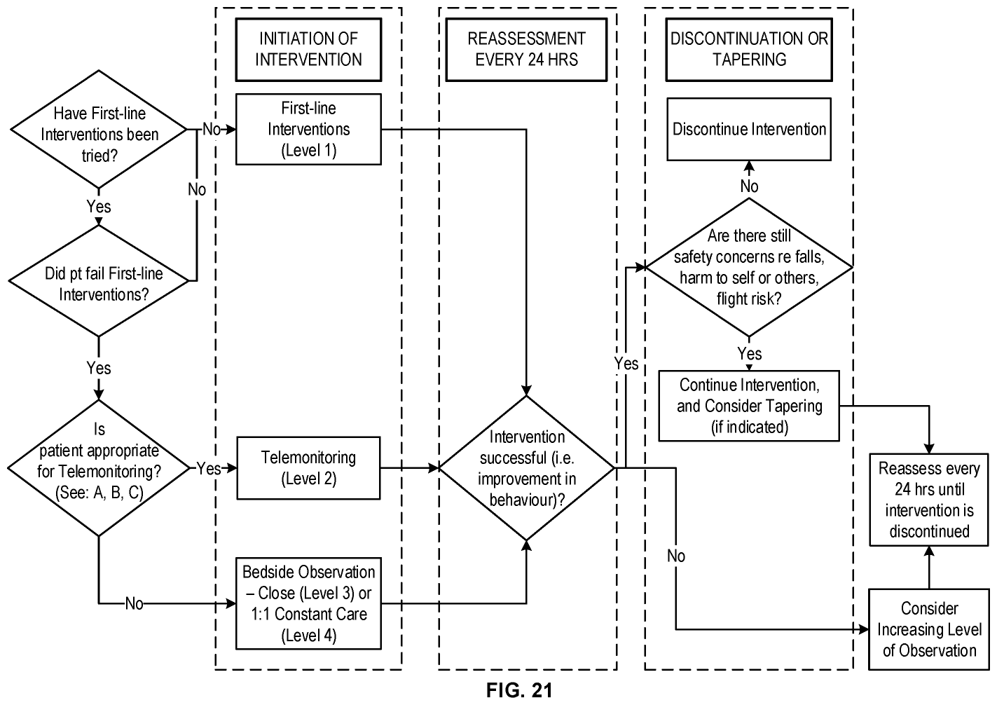

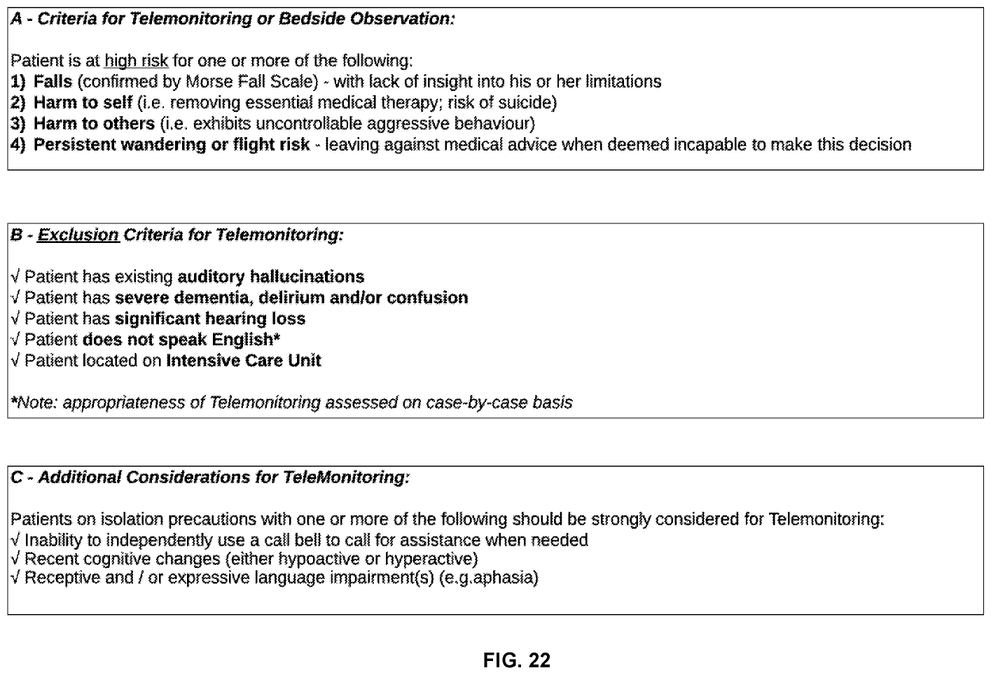

The systems and methods described above advantageously can be set up to comply with an implementation policy. The implementation of new technology within a healthcare setting requires extensive planning, especially if it requires a change in clinical practice. The use of bedside constant observation for patients with delirium, dementia, and/or confusion has been the standard of care at most healthcare organizations. Remote patient monitoring adds a new dimension in patient observation and changes the approach clinicians take when managing these types of patients. Clear criteria and guidelines are required to provide clinicians with appropriate decision-making support to make a smooth transition to changes in patient care. Referring now to FIG. 21 and FIG. 22, shown therein is an example decision support tool for initiation, continuation, and discontinuation of remote monitoring, along with consideration factors for appropriateness of remote monitoring. A step-by-step approach allows clinicians to safely and independently decide on the most appropriate level of observation, with the goal to start with the least intrusive and most cost-effective strategies. Escalation to the next level of observation can be automated in accordance with the policy and associated procedures developed to guide clinicians' decision-making safely.

### Study:

To assess if Remote Patient Monitoring (RPM) can be implemented in a way that provides the constant observation function in a more cost-effective manner without compromising patient safety.

In the study, clinical data was obtained for the following: (1) Fall Rates, (2) Adverse Events, (3) Lung Transplant Mortality Rate and (4) Constant 1:1 Bedside Hours and Cost. The study was conducted on patients at inpatient units at three different hospitals (Hospitals A, B and C) with start dates ranging from July 2016 to August 2019.

### Implementation Timelines:

The study was implemented under the following timelines:
Initial pilot technical setup July 2016 to December 2016—Thoracic Surgery & Respirology Inpatient Unit at Hospital A.
Phased approach across all inpatient units at Hospital A from January 2016 to April 2018 to align infrastructure, digital executive teams.
April 2018—went live with RPM on all inpatient units at Hospital A.
August 2019—went live with RPM on all inpatient units at Hospitals B and C.

### Patient Demographics

There was a total of 1,295 patients remotely monitored in the study pilot from July 2016 to December 2019. There were 53 (4%) young adults (18-40 years of age), 345 (28%) middle-aged adults (40-65 years of age) and 829 (68%) older adults (65 years of age or older). Of the 1,295 patients, 40% were females and 60% were males. Approximately half of the patients were surgical (n=710, 55%) who were recovering from their surgery while in hospital. The other half were either transplant patients having recently undergone transplantation or admitted to hospital while waiting for transplant (n=272, 21%) or patients admitted for medical conditions (n=313, 24%), such as pneumonia, sepsis, etc.

In order for a patient to qualify for continuous observation, they must have met one of the following inclusion criteria:
High Risk for Falls (using Morse Fall Scale) and lack of insight into his/her limitations;
High Risk for Harm to Self (i.e., removing essential medical therapy; risk of suicide);
High Risk for Harm to Others (i.e., exhibits uncontrollable aggressive behavior towards medical staff or other patients);
Persistent Wandering or Flight Risk (i.e., leaving against medical advice when deemed incapable to make this decision).

Once it was established that the patient required continuous monitoring, the observer followed the clinical pathway decision tools to assist in deciding the level of continuous observation the patient required. The options were: 1) No continuous monitoring needed and only conservative interventions (e.g., bed alarm, socks with grip bottoms); 2) RPM; or 3) 1:1 Bedside Continuous Observation.

Within the study, the main reason that most patients were remotely monitored was found to be multi-factorial. The most common combination was High Risk for Falls and High Risk for Self Harm (such as pulling off medically necessary oxygen masks, drains, tracheostomy tube, etc.). Patients who were wanderers or high risk for harming others typically also had other risk factors associated with them (e.g., high risk for falls, high risk for harming themselves) and therefore often fell into the combination category. There were very few individuals that were solely monitored due to risk of harming others or high risk for wandering. Table 6 below illustrates these findings.

TABLE 6
Reasons for Remote Monitoring

| Remotely Monitoring Categories | Number of Patients N (%) | |
|---|---|---|
| High Risk of Falls | 275 | (21%) |
| High Risk of Self Harm | 122 | (9%) |
| High Risk of Harm to Others | 1 | (0%) |
| Wandering | 9 | (1%) |
| Combination of the above categories | 909 | (69%) |

### Fall Rates:

Table 7 below shows fall rates for all three sites both pre- and post-implementation. Fall rates at Hospitals A, B, and C were reported/calculated for falls with injury per 10,000 adjusted patient days rolling over the last 12 months per hospital site. The RPM program allowed nursing staff to replace a physical person (a resource-intensive & expensive approach at $25/hour per patient) from the bedside who can continuously monitor the patient 1:1 with remote video technology (a more cost-efficient option at $8.90/hr per patient) and remotely redirect patients with voice to not engage in risky behavior that could potentially cause a fall or some form or harm. The general trend post-implementation of RPM was a decline in fall rates.

TABLE 7
Fall rates for all three hospital sites
both pre and post implementation

| | FY 16/17 | FY17/18 | FY 18/19 | FY 19/20 YTD Oct |
|---|---|---|---|---|
| All Acute Sites | 0.18 | 0.23 | 0.15 | 0.09 |
| Hospital A | 0.22 | 0.26 | 0.13 | 0.08 |
| Hospital B | 0.08 | 0.30 | 0.22 | 0.00 |
| Hospital C | 0.25 | 0.13 | 0.13 | 0.19 |

### Adverse Events:

Adverse events were reviewed post RPM implementation from Apr. 1, 2017, to Mar. 31, 2018 (at the surgical and medicine inpatient units at Hospital A). The incident reports that were completed for patients with bedside 1:1 constant observation (the current standard of care) were compared with the approach of RPM. Front-line nursing staff was provided with clinical decision pathways that guided the user through various decision points required to determine if the patient required continuous observation and whether RPM was indicated (instead of 1:1 bedside monitoring). These clinical decision pathways are depicted in FIGS. 21 and 22. The data showed that there were less falls and less adverse events in the RPM group compared to the Bedside 1:1 group (the standard of care). The adverse events included: pulling out an IV, a chest tube, a foley catheter, etc. The conclusion was that the clinical decision pathways developed were accurate in determining which patients were appropriate for RPM. The study demonstrated that the RPM system did not have a higher incidence of falls or adverse events despite removing a physical person at the patients' bedside. Table 8 shows the adverse events by type occurring during RPM and 1:1 bedside monitoring.

TABLE 8
Adverse Events at Hospital A

| Types | RPM | Bedside 1:1 |
|---|---|---|
| Fall | 7 | 25 |
| IV Pulled Out | 3 | 0 |
| Oxygen Pulled Off/Desaturation | 1 | 0 |
| NG Pulled Out | 1 | 0 |
| Chest tube Pulled Out | 1 | 0 |
| Foley Pulled Out | 0 | 2 |
| Picc Pulled Out | 0 | 1 |
| Patient Elopement | 0 | 1 |
| Total | 13 | 29 |

### Pre Lung Transplant Mortality Rate:

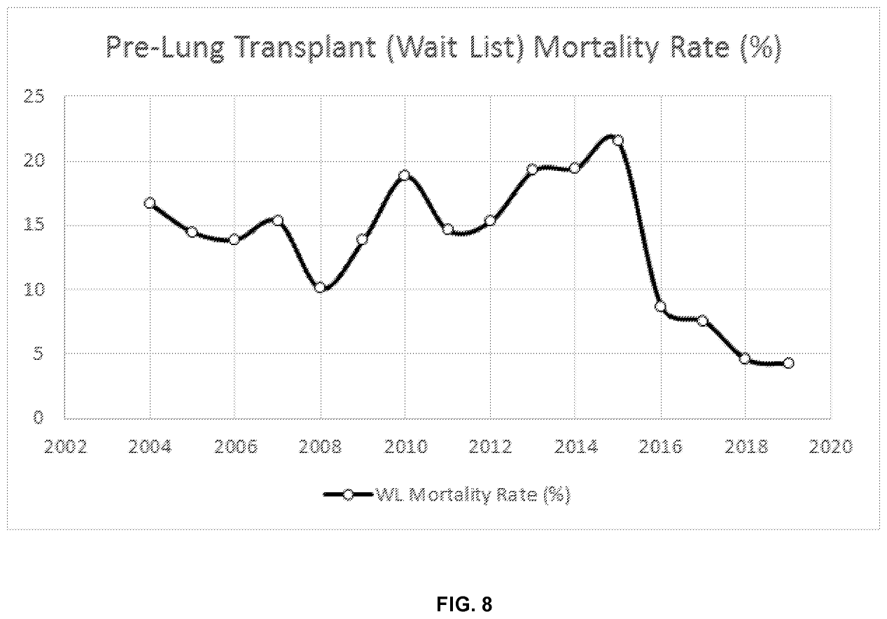

Table 9 shows the mortality rate of pre-lung transplant patients. Patients who are candidates for lung transplantation typically wait outside of the hospital for a potential compatible donor. However, there is a small population of patients who are too sick to wait at home typically due to high oxygen requirements and must be admitted to hospital while waiting for potential lung transplantation. There was a trend where the mortality rate of the pre-lung transplant patients was increasing over time, with an all-time high in 2015. In 2016, RPM was implemented, and the pre-lung transplant mortality rate decreased from 21% to 9%. In 2019, the mortality rate was 4% the lowest mortality rate to date. In particular, Table 9 shows the number of lung transplants by year and the corresponding Wait List (WL) mortality rate during the period of 2004 to 2019. The pre-lung transplant Wait List mortality rate is shown graphically in FIG. 8, where the decline in mortality rate following implementation of RPM in 2016 is visually apparent.

TABLE 9
Pre Lung Transplant Mortality Rate

| Year | # of Lung Transplants | WL Mortality Rate (%) |
|---|---|---|
| 2004 | 64 | 16.67 |
| 2005 | 68 | 14.46 |
| 2006 | 87 | 13.89 |
| 2007 | 100 | 15.32 |
| 2008 | 85 | 10.08 |
| 2009 | 102 | 13.83 |
| 2010 | 84 | 18.80 |
| 2011 | 102 | 14.60 |
| 2012 | 104 | 15.33 |
| 2013 | 133 | 19.25 |
| 2014 | 115 | 19.44 |
| 2015 | 128 | 21.48 |
| 2016 | 145 | 8.70 |
| 2017 | 170 | 7.57 |
| 2018 | 197 | 4.59 |
| 2019 | 212 | 4.25 |

### Bedside Constant Observer Hours & Cost:

A primary goal for RPM is to reduce bedside 1:1 constant observation by replacing it (where applicable) with RPM instead. The cost of bedside 1:1 observation has increased over the years (including during the pilot). When the pilot was initially started, it cost $18/hr for a bedside constant observer to monitor one patient; after the pilot concluded, it ranged between $23-25/hr. With RPM, healthcare providers are able to change the ratio of the constant observer from 1:1 to 1:6 or 1:8, providing a more cost-efficient way to continuously monitor patients.

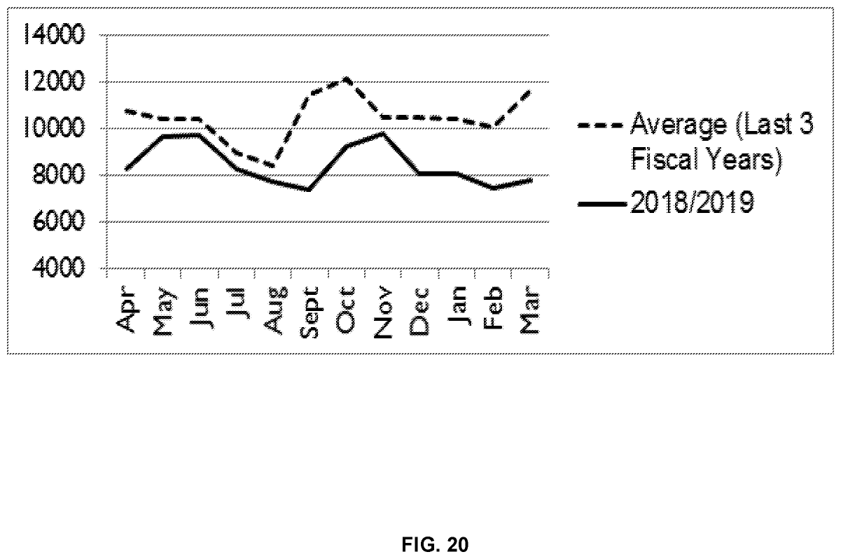

FIG. 20 shows a chart of the bedside constant observer hours in the 2018-2019 fiscal year (i.e., April 2018 to March 2019) compared to the average usage during the three-year period of April 2014 to March 2017 prior to implementation of RPM. There is a great variation in usage of bedside constant observation at all three hospitals (Hospital A, B, and C) from day-to-day, month-to-month, and year-to-year. However, the general trend was that the need of bedside 1:1 constant observation was increasing over time and driving costs associated with it substantially upwards.

Therefore, 1:1 bedside continuous observation usage post-implementation of RPM was compared with the average 1:1 bedside continuous observation usage during the three-year period of April 2014 to March 2017 prior to implementation of RPM. Post-implementation, there was a general decline in bedside 1:1 constant observation usage, which resulted in a cost avoidance of $800,000.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### REFERENCES

(1) Statistics Canada, (2017). Annual Demographic Estimates: Canada, Provinces and Territories. Analysis: Population by age and sex. Retrieved from: https://www150.statcan.gc.ca/nl/pub/91-215-x/2017000/sec2-eng.htm
(2) Government of Canada, (2014). Action for Seniors Report. Retrieved from: https://www.canada.ca/en/employment-social-development/programs/seniors-action-report.html
(3) Rochefort, C. M., Ward, L., Ritchie, J. A., Girard, N., & Tamblyn, R. M. (2011). Patient and nurse staffing characteristics associated with high sitter use costs. Journal of Advanced Nursing, 24, 1-10.
(4) Votruba, L., Graham, B., Wisinski, J., & Syed, A. (2016). Video monitoring to reduce falls and patient companion costs for adult inpatients. Nursing Economics, 34(4), 185-189.
(5) Jeffers, S., Searcey, P., Boyle, K., Herring, C., Lester, K., Goetz-Smith, H., & et al. (2013). Centralized video monitoring for patient safety: A denver health lean journey. Nursing Economics, 31(6), 298-306.
(6) Burston, P. L., & Vento, L. (2015). Sitter reduction through mobile video monitoring. The Journal of Nursing Administration, 45(7/8), 363-369.
(7) Cypel, M., Yeung, J., Liu, M., Anraku, M., Chen, F., Karolak, W., et al (2011). Normothermic Ex Vivo Lung Perfusion in Clinical Lung Transplantation. New England Journal of Medicine, 364, 1431-1440.

1. A computer-implemented method of managing a remote patient monitoring (RPM) system, wherein the method is implemented by a central server, an RPM client, and a networked monitoring device, and comprises:
initializing the RPM system for remote monitoring of at least one patient location using a network and a networked monitoring device;
receiving video data and physiological data over the network for the at least one patient location from the networked monitoring device;
transmitting 2-way audio data over the network for the at least one patient location;
displaying at least one viewport at the RPM client, the at least one viewport showing the video data and the physiological data for the at least one patient location;
automatically detecting a patient situation requiring attention in the at least one patient location and indicating the patient situation on the RPM client; and
receiving input at the RPM client of a response to the patient situation.

2. The method of claim 1, wherein initializing the RPM system further comprises:
receiving a request from the RPM client to set up a user interface and display the user interface;
providing camera data to the RPM client;
updating the available/monitoring camera list with available and monitoring cameras;
receiving a request from the RPM client to set up a viewport to monitor a specific camera from the available/monitoring camera list;
connecting the RPM client to the specific camera using real-time streaming protocol (RTSP);
receiving video frames from the specific camera and showing the video frames in the viewport;
performing an adjustment of the specific camera according to a request from the RPM client where the adjustment includes adjusting one of a pan, tilt, and/or zoom setting for the specific camera; and
sending audio input to a patient speaker associated with the specific camera where the audio input is received at the RPM client.

3. The method of claim 1, wherein the detected patient situation is that a patient is engaging in self harm and the method further comprises:
signaling to the RPM client to instruct a tele-monitor to attempt to redirect the patient verbally;
determining whether the redirection was successful; and
when the redirection is not successful:
signaling to the RPM client to instruct the tele-monitor to contact an assigned nurse to attend to the patient; and
when the assigned nurse does not receive the contact, signaling to the RPM client to instruct the tele-monitor to contact a nursing station to attend to the patient; and
determining that the patient situation has been responded to by receiving an indication that the assigned nurse or the nursing station has attended to the patient.

4. The method of claim 1, wherein the detected patient situation is that an SpO₂ level for a patient has dropped below an SpO₂ threshold level and the method further comprises:
signaling to the RPM client to instruct the tele-monitor to contact an assigned nurse to attend to the patient;
when the assigned nurse does not receive the contact, signaling to the RPM client to instruct the tele-monitor to contact a nursing station to attend to the patient; and
determining that the patient situation has been responded to by receiving an indication that the assigned nurse or the nursing station has attended to the patient.

5. The method of claim 1, further comprising:
receiving video frames from a given camera;
defining a reference background image including the patient from the video frames and defining a current image with the patient, the reference background image comprising background image pixels and the current image comprising current image pixels;
creating a background model using the background image pixels using a mixture of Gaussian distributions, the background model having a background model distribution;
classifying the current image pixels in the current image as background pixels or foreground pixels by calculating how close the current image pixels are from the background model distribution via Mahalanobis distance;
collecting the current image pixels classified as foreground pixels to generate a foreground image;
applying a median blur filter to the foreground image to obtain a first filtered foreground image;
applying a threshold filter to the first filtered foreground image to obtain a second filtered foreground binary image;
applying an erosion filter to the second filtered foreground binary image to obtain a third filtered foreground binary image;
applying a dilation filter to the third filtered foreground binary image to obtain a fourth filtered foreground binary image, the fourth filtered foreground binary image comprising 0-regions and 1-regions;
finding borders of the 1-regions in the fourth filtered foreground binary image to generate contours;
finding the contours that have areas larger than a predefined sensitivity value thereby defining found contours;
overlaying the found contours onto the current image to obtain an overlaid current image; and
displaying the overlaid current image at the RPM client.

6. The method of claim 1, further comprising:
receiving a video frame from a given camera;
selecting a trained machine learning model for determining probabilities for pixels being associated with different classes in the video frame;
calculating pixel class probabilities from the video frame using the trained machine learning model;
assigning a pixel class label to each pixel using a highest class probability determined for each pixel;
extracting class regions based on connected pixels that have the same pixel class label;
calculating a bounding box around the connected regions;
finding motion contours that have areas larger than a predefined sensitivity value thereby defining found motion contours;
masking the found motion contours for bounding boxes for bed and person classes thereby defining a masked motion contour;
overlaying the masked motion contour for a person on the video frame to obtain an overlay image; and
displaying the overlay image at the RPM client.

7. The method of claim 6, wherein the trained machine learning model is an artificial neural network that is trained by supervised learning over datasets obtained from video data stored at the RPM system.

8. The method of claim 1, wherein the incident comprises a patient falling out of bed and the method comprises use machine learning methods to predict when the incident will take place based on the video data received at the RPM client or the incident comprises a low patient SpO₂ level below an SpO₂ threshold, and the method comprises use machine learning methods to predict when the incident will take place based on the physiological data received at the RPM client.

9. (canceled)

10. The method of claim 1, further comprising:
receiving gaze data from the RPM client on a gaze direction of the tele-monitor determined using an eye tracker, the gaze data including gaze direction vectors;
performing screen calibration of a screen of the RPM client;
calculating a screen pixel location from the gaze direction vectors;
identifying when the gaze direction is outside of the viewport based on the screen pixel location; and
when the gaze direction is outside of the viewport longer than a gaze alert timer threshold, providing an audio and or video alert to the tele-monitor to prompt the tele-monitor to view the viewport, and optionally
translating between first speech input received by the RPM client and second speech input received from the at least one patient location using natural language processing, speech recognition, and speech synthesis so that communication at the RPM client and the least one patient location is in different languages spoken by individuals at both the RPM client and the least one patient location.

11.-15. (canceled)

16. A system for remote patient monitoring (RPM), the system comprising:
a server comprising a data store and at least one processor coupled to the data store;
an RPM client that is a software program that is executed by a computing device that is connected to the server via a network; and
a networked monitoring device that is connected to the server and the computing device having the RPM client via the network;
wherein the server is configured to initialize the RPM system for remote monitoring of at least one patient location using the network, and
wherein the RPM client is configured to
receive video data and physiological data over the network for the at least one patient location via the networked monitoring device;
transmit 2-way audio data over the network for the at least one patient location;
display at least one viewport at the RPM client, the at least one viewport showing the video data and the physiological data for the at least one patient location;
automatically detect a patient situation requiring attention in the at least one patient location and indicate the patient situation on the RPM client; and
receive input from the RPM client of a response to the patient situation.

17. The system of claim 16, wherein the server is configured to initialize the RPM system by:
receiving a request from the RPM client to set up a user interface and display the user interface;
providing camera data to the RPM client;
updating the available/monitoring camera list with available and monitoring cameras;
receiving a request from the RPM client to set up a viewport to monitor a specific camera from the available/monitoring camera list;
connecting the RPM client to the specific camera using real-time streaming protocol (RTSP);
receiving video frames from the specific camera and showing the video frames in the viewport;
performing an adjustment of the specific camera according to a request from the RPM client where the adjustment includes adjusting one of a pan, tilt, and/or zoom setting for the specific camera; and
sending audio input to a patient speaker associated with the specific camera where the audio input is received at the RPM client.

18. The system of claim 17, wherein the detected patient situation is that a patient is engaging in self harm and the computing device is configured to execute instructions to:
provide a signal at the RPM client to instruct a tele-monitor to attempt to redirect the patient verbally;
determine whether the redirection was successful; and
when the redirection is not successful:
signal to the RPM client to instruct the tele-monitor to contact an assigned nurse to attend to the patient; and
when the assigned nurse does not receive the contact, signal to the RPM client to instruct the tele-monitor to contact a nursing station to attend to the patient; and
determine that the patient situation has been responded to by receiving an indication that the assigned nurse or the nursing station has attended to the patient.

19. The system of claim 16, wherein the detected patient situation is that an SpO₂ level for a patient has dropped below an SpO₂ threshold level and the computing device is configured to execute instructions to:
signal to the RPM client to instruct the tele-monitor to contact an assigned nurse to attend to the patient;
when the assigned nurse does not receive the contact, signal to the RPM client to instruct the tele-monitor to contact a nursing station to attend to the patient; and
determine that the patient situation has been responded to by receiving an indication that the assigned nurse or the nursing station has attended to the patient.

20. The system of claim 16, wherein the computing device is configured to execute instructions to:
receive video frames from a given camera;
define a reference background image including the patient from the video frames and defining a current image with the patient, the reference background image comprising background image pixels and the current image comprising current image pixels;
create a background model using the background image pixels using a mixture of Gaussian distributions, the background model having a background model distribution;
classify the current image pixels in the current image as background pixels or foreground pixels by calculating how close the current image pixels are from the background model distribution via Mahalanobis distance;
collect the current image pixels classified as foreground to generate a foreground image;
apply a median blur filter to the foreground image to obtain a first filtered foreground image;
apply a threshold filter to the first filtered foreground image to obtain a second filtered foreground binary image;
apply an erosion filter to the second filtered foreground binary image to obtain a third filtered foreground binary image;
apply a dilation filter to the third filtered foreground binary image to obtain a fourth filtered foreground binary image, the fourth filtered foreground binary image comprising 0-regions and 1-regions;
find borders of the 1-regions in the fourth filtered foreground binary image to generate contours;
find the contours that have areas larger than a predefined sensitivity value thereby defining found contours;
overlay the found contours onto the current image to obtain an overlaid current image; and
display the overlaid current image at the RPM client.

21. The system of claim 16, wherein the computing device is configured to execute instructions to:
receive a video frame from a given camera;
select a trained machine learning model for determining probabilities for pixels being associated with different classes in the video frame;
calculate pixel class probabilities from the video frame using the trained machine learning model;
assign a pixel class label to each pixel using a highest class probability determined for each pixel;
extract class regions based on connected pixels that have the same pixel class label;
calculate a bounding box around the connected regions;
find motion contours that have areas larger than a predefined sensitivity value thereby defining found motion contours;
mask the found motion contours for bounding boxes for bed and person classes thereby defining a masked motion contour;
overlay the masked motion contour for a person on the video frame to obtain an overlay image; and
display the overlay image at the RPM client.

22. The system of claim 21, wherein the trained machine learning model is an artificial neural network that is trained by supervised learning over datasets obtained from video data stored at the RPM system.

23. The system of claim 16, wherein the incident comprises a patient falling out of bed and the computing device is configured to execute machine learning methods to predict when the incident will take place based on the video data received at the RPM client or the incident comprises a low patient SpO₂ level below an SpO₂ threshold, and the computing device is configured to execute machine learning methods to predict when the incident will take place based on the physiological data received at the RPM client.

24. (canceled)

25. The system of claim 16, wherein the computing device is configured to execute instructions to:
receive gaze data from the RPM client on a gaze direction of the tele-monitor determined using an eye tracker, the gaze data including gaze direction vectors;
perform screen calibration of a screen of the RPM client;
calculate a screen pixel location from the gaze direction vectors;
identify when the gaze direction is outside of the viewport based on the screen pixel location; and
when the gaze direction is outside of the viewport longer than a gaze alert timer threshold, provide an audio and or video alert to the tele-monitor to prompt the tele-monitor to view the viewport, and optionally
translate between first speech input received by the RPM client and second speech input received from the at least one patient location using natural language processing, speech recognition, and speech synthesis so that communication at the RPM client and the least one patient location is in different languages spoken by individuals at both the RPM client and the least one patient location.

26. (canceled)

27. The system of claim 16, wherein the at least one networked monitoring device comprises at least one of a locator that is used to configure a subnet for the patient locations at one physical location and a mobile patient monitoring cart that is used to create its own subnet to connect to the network and the mobile patient monitoring cart comprises a camera, a speaker, and at least one physiological measuring device incorporated into one mobile unit and the mobile patient monitoring cart is deployed to a different patient location.

28. (canceled)

29. The system of claim 16, wherein the system further comprises multiple networked monitoring devices and multiple RPM clients to scale the remote monitoring to cover patient locations in different locations within one building or in different locations in different buildings including a patient home, and/or wherein the network comprises at least one of a wired subnet and a wireless subnet that uses at least one of dynamic IP and static IP.

30. (canceled)
